(19) United States
(12) Patent Application Publication
(10) Pub. No.: US 2025/0228463 A1
(43) Pub. Date: Jul. 17, 2025

(54) SYSTEMS AND METHODS FOR DYNAMIC ANALYSIS OF TISSUE VIABILITY USING OPTICAL IMAGING

(71) Applicants: Thomas Bruce Ferguson, JR., Raleigh, NC (US); Sunghan Kim, Winterville, NC (US)

(72) Inventors: Thomas Bruce Ferguson, JR., Raleigh, NC (US); Sunghan Kim, Winterville, NC (US)

(21) Appl. No.: 19/018,649

(22) Filed: Jan. 13, 2025

Related U.S. Application Data
(60) Provisional application No. 63/728,299, filed on Dec. 5, 2024.
(60) Provisional application No. 63/620,794, filed on Jan. 13, 2024.

(51) Int. Cl. A61B 5/024

(52) U.S. Cl. CPC A61B 5/02427

(57) ABSTRACT

Methods, systems, and computer-readable media for assessing tissue perfusion and viability are disclosed. The methods can analyze optical imaging data, including reflectance captured using various light sources and imaging modalities. Tissue perfusion and viability can be evaluated by incorporating spatial, temporal, and physiological parameters. These approaches can enable assessments across a variety of clinical and research contexts, supporting diagnostics, therapy, and monitoring.

### RELATED APPLICATIONS

This application claims the benefit of U.S. Provisional Application No. 63/728,299, filed on Dec. 5, 2024, and U.S. Provisional Application No. 63/620,794, filed on Jan. 13, 2024, the each of which is hereby incorporated by reference in its entirety.

### FIELD

The present disclosure generally relates to medical imaging and diagnostic technologies and, more particularly, to analyzing tissue properties and physiological parameters through optical imaging techniques.

### BACKGROUND

Assessing tissue health and functionality can be an important aspect of medical diagnostics and treatment planning. Parameters such as blood flow, oxygenation levels, and structural properties of tissues can often provide valuable information for identifying medical conditions, monitoring recovery, and guiding interventions.

Local tissue viability (LTV) can involve the evaluation of blood flow, perfusion, and oxygenation within tissues. A more detailed assessment of these physiological parameters may be achieved by use of advanced physiological and engineering models. LTV assessments can utilize optical methods in clinically relevant tissues, where multiple wavelengths of light illuminate the tissue and reflected light is captured for subsequent analysis.

Non-invasive optical imaging techniques, such as Laser Speckle Contrast Imaging (LSCI), Hyperspectral Imaging (HSI), and Near-Infrared Fluorescence (NIRF), can be used to evaluate these parameters. These methods can rely on the interaction of light with tissues to capture data related to blood flow dynamics, metabolic activity, and tissue characteristics. While multiple technologies may accomplish reflectance capture, transforming these reflectance data into accurate quantification of blood flow and perfusion can still present challenges and opportunities for improvement.

These technologies, while valuable in many contexts, may encounter certain challenges in specific applications. For instance, sensitivity to motion, limitations in spatial or temporal resolution, and the complexity of data analysis can impact their performance. Additionally, combining multiple physiological measurements into a cohesive and scalable framework can sometimes be difficult. There is ongoing interest in improving the ability to perform real-time assessments of tissue viability by integrating physiological parameters and refining the accuracy of perfusion quantification.

### SUMMARY

Some embodiments of the present disclosure relate to methods and systems for assessing tissue perfusion by incorporating dynamic physiological characteristics directly into the quantification process. These methods can utilize light interactions with clinically relevant tissues in the wavelength range of approximately 400-1000 nm to capture absorption, quenching, scattering, and reflection characteristics that may vary depending on the tissue and wavelength. This captured data can provide insights into tissue viability; however, the analysis can be computationally demanding, particularly for real-time applications, and may face certain challenges in implementation.

Various approaches can be used to analyze tissue perfusion, including techniques such as laser speckle contrast analysis (LASCA), laser speckle contrast imaging (LSCI), hyperspectral imaging (HSI), laser Doppler imaging (LDI), and near-infrared fluorescence imaging (NIRF). Each of these approaches can offer valuable insights in different contexts but may also encounter challenges related to motion artifacts, computational complexity, acquisition speed, or invasiveness. Additionally, some approaches may not fully account for the dynamic physiological processes underlying tissue perfusion, such as cardiovascular energetics and function, vascular hemodynamics and metabolism, and tissue structure. Addressing these physiological factors can be important for enhancing the relevance and accuracy of tissue perfusion quantification.

Some inventive concepts described herein can address these challenges by capturing the embedded dynamic physiology of flow and perfusion directly into the quantification methodology. These concepts can enable a comprehensive assessment of tissue viability, capturing a range of states from normal to non-viable. Unlike certain approaches that may rely on static or simplified models, the disclosed methods can dynamically quantify perfusion while accounting for periodicity, pulsatility, spatial variations, and temporal dynamics. This dynamic focus can improve the fidelity and accuracy of perfusion assessments, potentially providing actionable insights that are more aligned with the physiological context of the tissue.

Some embodiments can include a model for tissue perfusion assessment that can apply directly to raw imaging data generated by suitable imaging systems. This model can incorporate a set of validated attributes derived from raw data and associated analytics, enabling an assessment of tissue perfusion across various conditions. By integrating these attributes into the quantification process, some inventive concepts described herein can offer enhanced temporal and spatial granularity, improving precision and utility in clinical and diagnostic applications.

By basing perfusion quantification on dynamic physiological parameters, these methods can also allow integration with other assessments of tissue viability, such as evaluations of tissue oxygenation. This combined approach can be particularly valuable for assessing conditions where the interaction between tissue perfusion and oxygenation is diagnostically or therapeutically significant. Such conditions may include both transient and chronic impairments of tissue viability.

In some implementations, the methodologies and systems described herein can be collectively referred to as “DyPERF” (Dynamic Perfusion Evaluation and Representation Framework). DyPERF can represent an inventive framework for assessing tissue perfusion that integrates dynamic physiological aspects directly into the quantification process. Unlike traditional methods, which may rely on static or simplified measurements, the DyPERF framework can emphasize a dynamic and physiologically informed approach, which may offer enhanced accuracy and clinical relevance in tissue perfusion assessment.

The DyPERF can incorporate physiological variations, such as pulsatile blood flow, vascular dynamics, and tissue-specific hemodynamics, into its calculations. These factors can be quantified using parameters like a K_{DyPERF} parameter (described in more detailed herein), which can reflect aspects of flow and perfusion dynamics. This speckle contrast parameter can be calculated as the mean light intensity divided by the standard deviation of light intensity, while retaining the full space and time characteristics of the raw speckle data. By applying these inventive concepts, the DyPERF can provide a strong representation of tissue perfusion beyond magnitude alone.

The DyPERF can nevertheless support detailed temporal and spatial assessments, offering insights at both large (e.g., 10×10 cm) and small (e.g., 1×1 mm) Fields of View (FOV). This approach can allow the contextualization of perfusion data within the tissue's functional state, supporting assessments across a spectrum of tissue viability, ranging from normal to non-viable states. In some cases, the DyPERF can integrate with other tissue viability metrics, such as oxygenation status, to enable a broader perspective on tissue health. This integration can be applied to various diagnostic and therapeutic applications, including evaluating tissue conditions, monitoring wound healing, and managing chronic conditions. By incorporating these inventive concepts, the DyPERF can provide a dynamic and adaptable approach for tissue perfusion assessment, with potential applications across a wide range of clinical and research settings.

Techniques for capturing, analyzing, and quantifying tissue perfusion and oxygenation dynamics in clinical tissues are disclosed. These techniques can involve the use of coherent and non-coherent light sources to illuminate tissues, with single or multi-sensor cameras capturing reflected light data to create analyzable datasets. The disclosed techniques can facilitate understanding and quantification of the flow and perfusion velocities of red blood cells, as well as the oxygenation parameters at vessel, arteriolar, and capillary levels of anatomy. The techniques can document and/or quantify normal and non-normal characteristics of tissue perfusion, oxygen delivery, and utilization, both spatially and temporally. These analyses can span fields of view and time intervals sufficient for clinical decision-making, enabling real-time assessments. Comparative evaluations of characteristics across tissues and regions of interest, and dynamic tracking of physiologic drivers, can also be performed without reliance on image formats.

### BRIEF DESCRIPTION OF THE DRAWINGS

Throughout the drawings, reference numbers can be re-used to indicate correspondence between referenced elements. The drawings are provided to illustrate embodiments of the present disclosure and do not to limit the scope thereof.

FIG. 1 illustrates a block diagram of a speckle data intake and processing system for acquiring, processing, organizing, and analyzing speckle data to assess tissue perfusion and associated physiological parameters.

FIG. 2 illustrates a data flow diagram that demonstrates an example process for acquiring, processing, and/or analyzing speckle data to derive tissue perfusion characteristics and related physiological insights.

FIG. 3 illustrates an example conceptual schematic of a Tissue Perfusion Assessment (TPA) model, which incorporates pre-K and post-K components centered around the laser speckle contrast 3D block (m×n×t).

FIG. 4 illustrates an engineering graphic representation of a potential TPA model, outlining the stepwise process from initial raw speckle data to the generation of advanced perfusion assessment metrics.

FIG. 5 illustrates an example of the DyPERF specific time-series data that can be obtained from three different regions of the human palm, recorded using three separate cameras.

FIG. 6 illustrates an example of dynamic changes in tissue perfusion during a controlled physical maneuver, specifically an isometric ball squeeze followed by a rest phase.

FIG. 7 illustrates an analysis in two different subjects (A and B) of perfusion magnitude determination comparison between traditional near-infrared laser speckle contrast imaging (NIR-LSCI) methods and the DyPERF approach, both analyzing the same raw speckle data.

FIG. 8 illustrates the application of the DyPERF methodology to quantify tissue perfusion dynamics vs. translational motion in a subject with chronic limb-threatening ischemia (CLTI).

FIG. 9 presents a detailed application of the DyPERF methodology to assess changes in tissue perfusion in a CLTI subject following a peripheral arterial lower extremity intervention.

FIG. 10 illustrates the relationship between the DyPERF quantification method and traditional cardiovascular physiologic parameters, specifically the Rate Pressure Product (RPP), in normal subjects at rest.

FIG. 11 illustrates the Spatial Domain Regional Area (RA) analysis of normal perfusion distribution across different hand segments, specifically the fingertips, proximal digits/palmar arch, and palm, in a normal adult volunteer.

FIG. 12 demonstrates the spatial RA analysis of acutely injured burn tissue on the medial hand and forearm.

FIG. 13 illustrates the co-location of analyzed spatial regional area (RA) on a still image from the spatial RA analysis video with their corresponding DyPERF time-series curves, emphasizing the integration of spatial and temporal perfusion analysis.

FIG. 14 demonstrates the combined temporal and spatial domain analysis of tissue perfusion using the DyPERF methodology in a post-versus pre-intervention (PvP) comparison format for a subject with injured bowel tissue undergoing a second-look procedure.

FIG. 15 demonstrates in normal tissue with normal perfusion the Distribution in Tissue Structure (DTS) analysis as an integrated component of the TPA model.

FIG. 16 illustrates the validation of the TPA model using data from the injured bowel case presented in FIG. 14.

FIG. 17 illustrates the spatial RA analysis of tissue perfusion, highlighting its ability to provide quantifiable insights at varying levels of spatial detail.

FIG. 18 demonstrates the DTS pattern analysis workflow, and the DTS sub-analysis which integrates the RA spatial graphic with a directionality histogram (DH) analysis to generate detailed insights into perfusion distribution and magnitude differences between regions of interest within the field of view (FOV) from a compromised bowel anastomosis in a GI surgical case.

FIG. 19 presents a further application of the DTS pattern analysis and sub-analysis, analyzing raw speckle data from a different clinical case involving burn injury.

FIG. 20 illustrates the reproducibility and utility of the TPA model in analyzing tissues with normal perfusion, highlighting consistent DyPERF magnitude curves, aligned RA spatial graphics, and uniform Directionality Histograms, while also highlighting subtle variations in non-perfused regions across the evaluated tissue segments.

FIG. 21 illustrates the detailed application of the TPA model in analyzing normal perfusion in normal tissues, focusing on the variability seen within a single cardiac cycle.

FIG. 22 illustrates the application of the TPA model to significantly diseased tissues with abnormal perfusion, demonstrating its utility in integrating perfusion magnitude and the distribution in tissue structure components for a comprehensive analysis.

FIG. 23 illustrates the application of DTS directionality sub-analyses in tissues at two stages of disease conditions, specifically pre- and post-intervention.

FIG. 24 demonstrates a clinical application of the TPA model, highlighting the directional convergence of the increase in perfusion magnitude (DyPERF) and the normalization of the DTS DH after a vascular intervention.

FIG. 25 provides a clinical example of divergence between the perfusion magnitude (DyPERF) and the DTS DH results direction of change following a vascular intervention.

FIG. 26 illustrates an example software design for implementing the TPA model.

FIG. 27 illustrates an example schematic for a software design focused on PVP analysis within the TPA model.

FIG. 28 illustrates the application of temporal scalability in analyzing DyPERF time-series data, highlighting the transformation of a 10-second interval of raw speckle-derived data into an idealized waveform that retains critical physiological and analytical features.

FIG. 29 illustrates the implementation of spatial scalability in tissue perfusion assessment.

### DETAILED DESCRIPTION

Methods for assessing tissue perfusion often rely on established technologies such as Laser Speckle Contrast Imaging (LSCI), Hyperspectral Imaging (HSI), Laser Doppler Imaging (LDI), and Near-Infrared Fluorescence (NIRF). While these techniques can be valuable for providing insights into blood flow and tissue oxygenation, they frequently face notable limitations. For example, LSCI can be sensitive to translational motion artifacts, which may distort data and reduce accuracy. HSI, while capable of capturing detailed spectral information, often involves high costs and computational complexity, making it less suitable for real-time clinical use. Similarly, LDI can be hindered by extended acquisition times that limit its practicality in dynamic surgical environments, and NIRF typically requires the administration of dyes, adding an invasive step to the procedure. Furthermore, traditional approaches often attempt to eliminate all motion artifacts during data processing, which, while simplifying the data, may inadvertently remove physiologically valuable information, such as the pulsatile dynamics of blood flow driven by cardiac cycles. This can reduce the clinical utility of the data by omitting key signals that may provide valuable insights into tissue perfusion.

Traditional methods for assessing tissue perfusion primarily focus on measuring flow magnitude—quantifying the amount of blood flow through vessels or in tissue regions. These methods often rely on analyzing isolated data points or individual frames, without incorporating contextual information from adjacent moments in space or time. Traditional laser speckle contrast analysis, for example, is executed by selecting a spatial, temporal, or definable intermediate window on which to base this quantification estimate of perfusion. While this approach simplifies data processing, it overlooks the continuous and dynamic nature of physiological processes, such as the pulsatile kinetic energy of blood flow driven by cardiac cycles. To further streamline data interpretation, traditional systems frequently aim to eliminate all motion artifacts. However, this can inadvertently discard critical physiological signals, including subtle variations tied to pulsatile dynamics. By simplifying the data in this way, traditional methods may fail to capture key physiological nuances, such as variations in tissue structure or localized perfusion patterns. This lack of granularity can limit their clinical utility, making it challenging to detect subtle yet clinically significant changes in dynamic medical environments.

Some inventive concepts described herein improve the process of assessing tissue perfusion by introducing moving observational windows into raw reflectance datasets. For example, unlike traditional LSCI analyses, raw speckle data can be converted to speckle intensity and derivatives using a moving observation window (e.g., 0.125 s), which allows space and time capture of physiological variation such as pulsatile blood flow.

Some inventive concepts described herein enhance tissue perfusion analysis by incorporating multiple attributes beyond flow magnitude. These include physiological dynamics, tissue structure, spatial distribution of perfusion, and temporal variations. For example, the system can analyze physiological attributes like heart rate or pulsatility, while simultaneously assessing spatial characteristics through localized perfusion patterns. This enables more detailed evaluations of tissue perfusion in clinical environments.

Some inventive concepts described herein introduce an alternative calculation for the speckle contrast parameter K. Unlike traditional approaches that calculate K as the ratio of standard deviation to mean intensity after selecting an analysis window, the disclosed methods calculate K (i.e., K_{DyPERF}) from the moving observational window speckle value at each pixel×pixel as the mean intensity divided by the standard deviation over the time of the entire laser speckle dataset. This preserves full temporal and spatial characteristics while reducing artifacts. This adjustment ensures a more robust and accurate representation of perfusion patterns in static and/or dynamic contexts.

Some inventive concepts described herein introduce the identification of the laser speckle contrast dataset as a three-dimensional block of speckle contrast data, combining directional and temporal dimensions for detailed analyses as a unit. For example, the system processes raw data to generate a block that includes information on tissue dimensions (e.g., horizontal, vertical) and temporal changes over time. By slicing this block along specific axes, analyses specific to the clinical scenario can evaluate localized perfusion dynamics, observe changes over time, and perform pre- and post-intervention comparisons.

Some inventive concepts described herein provide mechanisms for analyzing physiological dynamics such as heart rate, periodicity, and pulsatility. By applying temporal Fourier transforms to the magnitude time-series data, the system identifies subtle variations in physiological behavior that influence perfusion. For example, these analyses reveal underlying physiological changes that may not be apparent through traditional methods focused solely on magnitude.

Some inventive concepts described herein enable spatial analysis of tissue perfusion by dividing the three-dimensional block of data into defined regions. For example, clinicians can focus on specific areas of tissue to assess localized perfusion and compare those results with adjacent regions. This facilitates the identification of abnormalities, such as perfusion deficits, in a targeted and efficient manner.

Some inventive concepts described herein include a Directionality in Tissue Structure (DTS) analysis, which uses multidimensional Fourier transforms and anisotropic pattern analysis to evaluate the 3D block spatial and temporal organization of perfusion. For example, DTS analysis characterizes perfusion patterns in relation to tissue health, enabling clinicians to detect subtle irregularities or improvements in perfusion, both at baseline and following a treatment intervention.

### System Overview

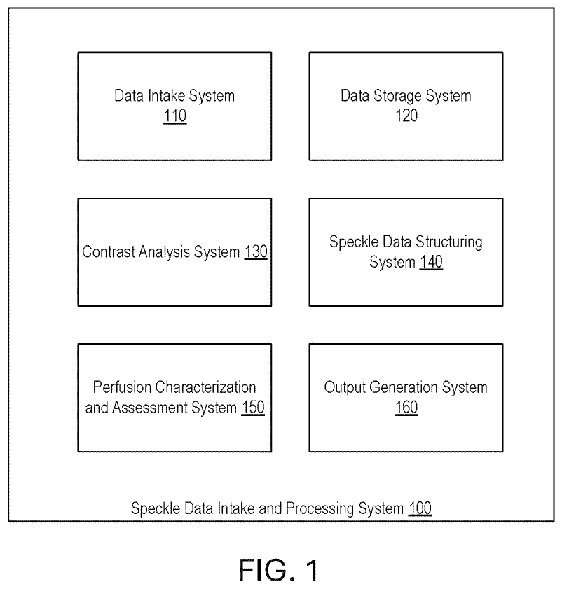

FIG. 1 illustrates a block diagram of a speckle data intake and processing system 100 for acquiring, processing, organizing, and analyzing speckle data to assess tissue perfusion and associated physiological parameters. The speckle data intake and processing system 100 can include a data intake system 110, a data storage system 120, a contrast analysis system 130, a speckle data structuring system 140, a perfusion characterization and assessment system 150, and an output generation system 160. Each component can operate independently or in conjunction with other components to facilitate efficient data acquisition and analysis. Although FIG. 1 depicts a single instance of each component, multiple instances of the same type of component can be deployed depending on system requirements.

The data intake system 110 can obtain raw speckle data, which represents light intensity measurements resulting from the interaction of light with tissue. The raw speckle data can serve as an input for assessing physiological parameters such as, but not limited to, blood flow, perfusion assessment, or tissue viability. Raw tissue oxygenation data can be concurrently captured using an accompanying illumination strategy. In some cases, the raw speckle data corresponds to a region of interest, which can refer to a specific area or multiple areas of tissue selected for analysis. A region of interest may include, but it not limited to, surface tissue (e.g., such as the skin), deeper anatomical structures like organs, or vascular networks on which perfusion evaluation is desired. For example, in a diagnostic setting, a region of interest could be a localized area suspected of compromised blood flow, while in a surgical context, a region of interest might span the entirety of a monitored surgical field.

The raw speckle data can be acquired across multiple spatial locations, providing detailed coverage of the region of interest. Spatial locations can correspond to individual points, pixels, or volumetric elements within a two-dimensional or three-dimensional imaging field. In some cases, the raw speckle data can correspond measurements over sequential time points, allowing for the observation of dynamic physiological processes, such as the pulsatile movement of blood driven by cardiac cycles or variations in tissue oxygenation during functional assessments. For example, the raw speckle data might include light intensity values for tens, hundred, thousands, tens of thousands, or hundreds of thousands of pixels in a high-resolution imaging grid, where each pixel captures a unique spatial location. These intensity measurements can be captured at intervals such as, but not limited to, milliseconds, seconds, minutes, hours, or days, creating a unique time-series dataset that reflects spatial and/or temporal variations.

The raw speckle data is obtained by the data intake system 110. For example, in some cases, the data intake system 110 may capture or receive the raw speckle data in the form of real-time or near real-time measurements. As another example, in some cases, the data intake system 110 may obtain the raw speckle data from stored datasets retrieved for retrospective analysis. Real-time data can be helpful in applications such as surgical monitoring, where continuous feedback on tissue perfusion enables immediate clinical decision-making. For instance, during surgery, the data intake system 110 might capture and process data to ensure sufficient blood flow to vital tissues, helping prevent ischemic damage. In some cases, previously recorded datasets may be retrieved to analyze historical trends, allowing clinicians to compare current measurements with past observations and assess changes in tissue health over time.

The structure of the raw speckle data can vary depending on the imaging system and the analysis requirements. For example, the raw speckle data can be composed of arrays or matrices of intensity values, where each entry represents the light intensity captured at a specific spatial location and time point. A three-dimensional dataset might be organized with axes corresponding to spatial dimensions (e.g., X and Y for surface imaging, or X, Y, and Z for volumetric imaging) and a temporal axis capturing the progression of intensity over time. This data structure can allow downstream systems to analyze spatial patterns, temporal dynamics, or both simultaneously.

The data storage system 120 can store raw speckle data, intermediate datasets, and/or related outputs in a structured and accessible manner. This storage can include light intensity and derivative values acquired by the data intake system 110, data generated by the contrast analysis system 130, the speckle data structing system 140, and/or the perfusion characterization and assessment system 150, and/or outputs such as speckle contrast datasets or perfusion analyses from the output generation system 160. For instance, the data storage system 120 can accommodate datasets ranging from small diagnostic snapshots to extensive datasets collected over time for clinical monitoring or research applications.

As described herein, the data storage system 120 can store speckle contrast datasets generated from raw speckle data. The speckle contrast datasets can represent a distribution of speckle contrast values, including, but not limited to, across multiple spatial locations within a region of interest over time. In some cases, the speckle contrast dataset can include a first dimension corresponding to a spatial axis, which can represent the region of interest, a second dimension corresponding to a temporal axis reflecting sequential time points, and/or a third dimension corresponding to speckle contrast values for each spatial location and time window. For example, such a dataset may include a multi-dimensional data cube, with each entry reflecting a contrast value linked to a specific location and temporal window, enabling detailed spatial and temporal domain analyses.

The data storage system 120 can accommodate short-term and/or long-term storage needs. For short-term scenarios, such as real-time monitoring, the data storage system 120 can temporarily store raw speckle data, speckle contrast datasets, and/or other related data for immediate processing by downstream systems. For example, during a surgical procedure, contrast values calculated in real time can be temporarily stored to support instantaneous analysis and decision-making. For longer-term applications, such as retrospective or longitudinal studies, the data storage system 120 can retain datasets for extended periods, supporting comparisons of pre- and post-treatment metrics or enabling evaluations of tissue perfusion changes over time.

The data storage system 120 can organize data in a manner that supports efficient access and retrieval. This organization may include multi-dimensional arrays or data cubes, where spatial dimensions (e.g., X and Y for surface imaging, or X, Y, and Z for volumetric imaging) are combined with a temporal dimension representing time-series data. Additional metadata, such as acquisition timestamps, imaging parameters, or contextual details about the region of interest, can also be stored alongside primary datasets. For instance, a dataset might include annotations describing the specific anatomical region or clinical context, aiding retrieval and interpretation in future analyses.

The data storage system 120 can employ various storage technologies to meet different operational needs. Volatile memory, such as RAM, may be used for high-speed, temporary storage during active imaging or processing tasks. For persistent storage, the data storage system 120 can utilize solid-state drives (SSDs), hard disk drives (HDDs), and/or distributed network storage. Cloud-based solutions can also be employed, enabling scalable, redundant storage accessible from multiple locations.

The data storage system 120 can include or be implemented as cloud storage, such as Amazon Simple Storage Service (S3), Elastic Block Storage (EBS) or CloudWatch, Google Cloud Storage, Microsoft Azure Storage, InfluxDB, etc. The data storage system 120 can be configured to provide high availability, highly resilient, low loss data storage. In some cases, to provide the high availability, highly resilient, low loss data storage, the data storage system 120 can store multiple copies of the data across the same or different types of data stores (e.g., solid state, hard drive, tape, etc.).

The contrast analysis system 130 can process raw speckle data to calculate speckle contrast values, providing a quantitative representation of physiological tissue characteristics such as blood flow and perfusion. The contrast analysis system 130 can obtain intensity data (e.g., raw speckle data) from the data intake system 110 or retrieve it from the data storage system 120. The intensity data can be organized by space or time or both at the pixel level, with each value representing light-tissue interactions at specific spatial locations and time points.

The contrast analysis system 130 can determine a series of speckle contrast values for one, some, or each spatial location within a region of interest. The calculation of speckle contrast values in DyPERF may not require the pre-definition of temporal, spatial, or spatio-temporal windows as with traditional LSCI analyses. Rather, the speckle contrast values in DyPERF can retain the full temporal and spatial characteristics of the moving observational window-based raw speckle data.

In some cases, with the speckle contrast calculated by the contrast analysis system 130, the full spatial and temporal characteristics available at the speckle contrast stage of analysis (i.e., as the 3D block) allows for subsequent spatial and/or temporal domain analyses to follow. In this way the dynamic physiology of flow and perfusion can be included in these analyses.

In some cases, each speckle contrast value can be calculated as a ratio of the mean intensity to the standard deviation of intensity values over the entire space and time of the laser speckle dataset. Such an approach may differ from traditional methods, which often use the standard deviation divided by the mean intensity. By inverting this calculation, the contrast analysis system 130 can preserve subtle variations in spatial and temporal dynamics while potentially mitigating the effects of noise and motion artifacts. Velocity is now directly related to speckle contrast, and thence to the quantified estimate of perfusion. This new K calculation is applied at the pixel×pixel level to the speckle data file series sampled by the moving observational window around each file, with optimization of observation window for perfusion magnitude assessment. This approach retains the full spatial and temporal capabilities of the raw speckle data and the laser speckle contrast data. For instance, in clinical scenarios, this approach can enhance the identification of localized perfusion irregularities that might not be evident with traditional methods.

The speckle contrast values generated by the contrast analysis system 130 can reflect patterns of tissue perfusion and flow dynamics. Higher speckle contrast values correspond directly to regions with faster and perhaps more uniform perfusion, while lower speckle contrast corresponds to reduced or stagnant perfusion in tissues that may be normal or severely abnormal. These contrast values can be organized into structured datasets that provide spatial and temporal mappings of tissue characteristics, enabling further interpretation and visualization.

The contrast analysis system 130 can analyze real-time and/or stored data. For real-time applications, such as intraoperative monitoring, the contrast analysis system 130 can process incoming intensity data to provide timely updates on tissue perfusion. For retrospective studies, stored intensity data can be retrieved from the data storage system 120 and processed to generate contrast datasets, enabling comparisons across different time points or conditions. For example, datasets from pre- and post-treatment sessions can be analyzed to evaluate the effects of an intervention.

The output of the contrast analysis system 130 can include structured datasets organized into multi-dimensional arrays or data cubes. These datasets can span spatial dimensions, such as X and Y for surface imaging or X, Y, and Z for volumetric imaging, along with a temporal dimension representing changes over time. Metadata, such as acquisition parameters and processing timestamps, can accompany the datasets to provide additional context. These speckle contrast datasets can be integrated with downstream systems, such as the speckle data structuring system 140, for further analysis and clinical use. By accommodating various configurations of temporal windows and calculation methods, the contrast analysis system 130 can support a broad range of clinical and research applications, tailored to specific requirements.

The speckle data structuring system 140 can transform the outputs of the contrast analysis system 130 into structured speckle contrast datasets, providing an organized representation of raw analytical data. This transformation arranges the data in a way that supports efficient retrieval, interpretation, and integration into clinical or research workflows. By converting raw outputs into structured, multi-dimensional formats, the speckle data structuring system 140 facilitates the analysis and practical use of speckle contrast data.

The speckle data structuring system 140 can arrange speckle contrast values into multi-dimensional arrays or data cubes, creating a representation that highlights spatial and temporal relationships within the data. These structured speckle contrast datasets can include spatial dimensions, such as X and Y for surface imaging or X, Y, and Z for volumetric imaging, along with a temporal dimension capturing sequential changes over time. For example, the system may create a three-dimensional dataset where each layer corresponds to contrast values at a particular time point, or a more detailed dataset incorporating contextual dimensions such as imaging conditions. This structured arrangement allows for the examination of patterns in tissue perfusion across both space and time. As an illustrative example, the system may organize data into the following data structure:

TABLE 1

| Spatial Location | Time [s] | Speckle Contrast Value |
|---|---|---|
| [0, 0] | 0 | 0.5 |
| [0, 0] | 1 | 0.6 |
| [0, 0] | 2 | 0.55 |
| . . . | . . . | . . . |
| [0, 0] | 9 | 0.58 |
| [1, 0] | 0 | 0.52 |
| [1, 0] | 1 | 0.63 |
| [1, 0] | 2 | 0.56 |
| . . . | . . . | . . . |
| [9, 9] | 9 | 0.61 |

This representation arranges speckle contrast values by spatial location (e.g., [X, Y] coordinates) and temporal point (e.g., seconds), enabling straightforward analysis of perfusion dynamics across regions of interest.

In some cases, the speckle data structuring system 140 can embed metadata to add contextual information to the datasets. Metadata can include details such as, but not limited to, acquisition settings, temporal resolution, timestamps, anatomical descriptions of the region of interest, or clinical annotations. For instance, a dataset produced during a medical procedure might include metadata describing the imaging device used, the region of tissue analyzed, or the specific phase of the procedure. By embedding such information, the speckle data structuring system 140 transforms raw data into datasets that provide additional context and meaning, enhancing their usefulness for downstream applications.

The speckle data structuring system 140 can generate speckle contract datasets tailored for specific purposes, ensuring that the data can be effectively used across a variety of contexts. For example, the speckle data structuring system 140 may create structured data that integrates with visualization tools for displaying perfusion maps during clinical procedures, or may prepare datasets compatible with statistical or computational analysis frameworks for retrospective studies.

The speckle data structuring system 140 can improve accessibility by organizing and indexing datasets in ways that facilitate targeted retrieval. For example, data may be indexed by spatial location, time, or clinical context, allowing users to efficiently access relevant portions of the dataset without processing the entire collection. A researcher studying localized perfusion deficits, for instance, can query the system to extract data corresponding to a specific sub-region and time range, along with relevant metadata.

In some cases, the speckle data structuring system 140 can enhance the perception of the laser speckle contrast dataset as a three dimensional block (m×n×t), where m=the number of pixels in the horizontal direction, n=the number of pixels in the vertical direction, and t=the number of frames in the series or sub-series. Slices through the 3-D block at discrete time points, or time intervals, or of the block as a whole are used for further analyses.

The perfusion characterization and assessment system 150 can process data from the contrast analysis system 130 and/or the speckle data structuring system 140 to derive insights into tissue perfusion. The perfusion characterization and assessment system 150 can evaluate multiple dimensions of perfusion, including spatial, temporal, and/or structural characteristics, leveraging data inputs from earlier stages of the analysis pipeline.

The perfusion characterization and assessment system 150 can analyze data from the contrast analysis system 130, such K_{DyPERF} calculated speckle contrast values, to calculate perfusion magnitude. These values can be averaged across spatial locations and temporal windows to generate a series of magnitude values corresponding to the region of interest. Traditional LSCI analysis magnitude values can be plotted over time to reflect variations in perfusion determined by a spatial or temporal window. However, with the new DyPERF analyses, each dynamic magnitude value can be plotted as the unique time-series (incorporating the full spatial and temporal characteristics of the perfusion due to the optimized moving observational window). These DyPERF time-series can also be used to generate physiologic parameters and identify translational motion embedded in the original raw speckle data.

By interpreting structured datasets from the speckle data structuring system 140, the system can extend its analysis to the spatial and temporal domains within the data. Datasets from the three-dimensional block enable the perfusion characterization and assessment system 150 to evaluate localized perfusion metrics and dynamic temporal variations. For example, the perfusion characterization and assessment system 150 might analyze a specific subset of the structured data to identify changes in perfusion magnitude or to evaluate flow dynamics across spatial regions.

The perfusion characterization and assessment system 150 can perform temporal analysis by examining time-series data for patterns related to physiological processes, such as heart rate, periodicity, and/or pulsatility. Using methods such as Fourier transforms, the perfusion characterization and assessment system 150 can identify frequency components associated with cardiac oscillations or other rhythmic physiological events. For instance, heart rate can be calculated by isolating a frequency component corresponding to each cardiac cycle and converting it to beats per minute, while periodicity and pulsatility can be quantified to reflect consistency and amplitude variations, respectively.

Comparative analyses can allow the perfusion characterization and assessment system 150 to evaluate changes in perfusion metrics in the temporal domain, over time or across conditions. For example, the perfusion characterization and assessment system 150 can compare pre- and post-intervention data to quantify the effects of treatments or procedures. The perfusion characterization and assessment system 150 might calculate differences in perfusion magnitude values, highlight trends over successive time points, or assess perfusion uniformity before and after an event.

Spatial domain analyses can be equally important, as the perfusion characterization and assessment system 150 segments the region of interest into sub-regions to evaluate localized perfusion characteristics. By dividing the region into defined grids or areas, the perfusion characterization and assessment system 150 can calculate metrics for each sub-region, producing spatially resolved perfusion maps. These maps highlight variations in perfusion, identifying regions with higher or lower flow, which may indicate potential abnormalities or regions of interest for further investigation and the basis for tissue viability assessment.

The perfusion characterization and assessment system 150 can assess structural and directional characteristics of perfusion by analyzing patterns in the data. Multi-dimensional pattern analysis, using techniques such as multi-dimensional Fourier transforms, can evaluate anisotropic and isotropic features of tissue perfusion. For example, directionality histograms generated from this analysis can reveal the alignment and organization of perfusion patterns within tissue structures. These results can be compared to directionality data from normal tissues that are normally perfused, providing each TPA analysis with a normal perfusion distribution in tissue benchmark.

By processing inputs from both the contrast analysis system 130 and the speckle data structuring system 140, the perfusion characterization and assessment system 150 can evaluate various attributes of tissue perfusion including, but not limited to, magnitude, temporal dynamics (e.g., including periodicity, pulsatility, and/or heart rate), spatial distribution, directional properties, anatomical structure, or regional variations. These analyses support a wide range of clinical and research applications, enabling the perfusion characterization and assessment system 150 to provide detailed, actionable outputs tailored to specific needs. Examples include time-series graphs of perfusion dynamics, regional perfusion maps, and histograms summarizing directional features, all of which contribute to a comprehensive assessment of tissue perfusion.

The output generation system 160 processes data and results from upstream systems, including the contrast analysis system 130, the speckle data structuring system 140, and the perfusion characterization and assessment system 150. The output generation system 160 is configured to present data in a variety of formats tailored to specific end-users, such as clinicians, researchers, or automated decision-support systems, and can accommodate both clinical and investigative contexts.

The output generation system 160 can produce a range of outputs, including, but not limited to, numerical, visual, and textual formats, depending on the analysis performed and the intended application. For instance, the output generation system 160 can generate time-series graphs to illustrate temporal variations in perfusion magnitude, such as during a physiological event or following an intervention. These graphs can reflect patterns in tissue perfusion over time, offering insights into trends and helping identify potential anomalies.

Spatial outputs can be produced by the output generation system 160, such as color-coded perfusion maps (both video and still image) derived from datasets processed by the speckle data structuring system 140 and analyzed by the perfusion characterization and assessment system 150. These maps can segment regions of interest and visually highlight areas of reduced or irregular perfusion, aiding in the identification of ischemic or abnormal flow patterns. For example, spatial outputs might feature a grid-like segmentation of the region of interest with perfusion metrics overlaid for each section. Additional spatial domain outputs include spatial regional area (RA) time-series for analysis by the perfusion characterization and assessment system 150, and the time-series as a graphic for subsequent pattern analysis (see below).

For physiological analyses, the output generation system 160 can display results from advanced temporal evaluations, such as periodicity, pulsatility, and heart rate metrics derived from time-series data. These outputs can be represented as numerical summaries, frequency spectra, or graphical overlays that correlate temporal patterns with tissue perfusion dynamics. For example, a graph might depict periodic oscillations indicative of cardiac activity alongside calculated heart rate.

Additional spatial domain assessments can include directionality histograms or multi-dimensional plots generated through anisotropic and isotropic pattern analysis by the output generation system 160. These outputs can provide insights into the structural organization and directional characteristics of tissue perfusion, aiding in the evaluation of vascular flow alignment or identifying irregular directional patterns. Such analyses can assist in assessing tissue structure and vascular functionality.

Comparative analyses can be supported by the output generation system 160, with outputs highlighting differences between datasets. For instance, pre- and post-intervention comparisons can reveal improvements or persistent irregularities in tissue perfusion. These outputs might include side-by-side visualizations, percentage change calculations, or time-series graphs that depict variations across temporal and spatial dimensions.

Tailoring outputs to specific end-user requirements, the output generation system 160 can provide streamlined reports summarizing findings for clinicians or detailed datasets for researchers. A clinician might receive on screen in real-time a report containing a summary of numerical metrics, color-coded perfusion maps, and trend graphs to support quick decision-making. As another example, a researcher may access high-resolution visualizations, statistical analyses, and comprehensive metadata for further study.

Outputs from the output generation system 160 can be integrated into broader workflows, such as electronic health records (EHR) systems, where they may trigger alerts or recommendations based on detected abnormalities. The output generation system 160 can format outputs for compatibility with research databases, presentation software, or collaborative platforms, facilitating broader dissemination or interdisciplinary investigations.

The output generation system 160 can provide versatile and customizable outputs, including, but not limited to, time-series graphs and multi-dimensional Fourier transform (TFT)-based analyses; spatially resolved perfusion videos, maps and directionality histograms; pre- and post-intervention comparisons with percentage change metrics; multi-dimensional datasets integrating spatial and temporal insights; and/or statistical summaries and metadata for research purposes.

By synthesizing the results from upstream systems and transforming them into interpretable formats, the output generation system 160 supports applications such as clinical diagnostics, intraoperative monitoring, and post-intervention assessments. The output generation system 160 enhances the utility of complex analyses, enabling clear and actionable presentations of tissue perfusion insights for diverse end-users.

It will be appreciated that the speckle data intake and processing system 100 can incorporate a variety of software solutions to support its operations across different components, including but not limited to cloud-based platforms, computational libraries, and visualization tools. For example, the data storage system 120 may employ cloud-based platforms such as Amazon Simple Storage Service (S3), Elastic Block Storage (EBS), or Google Cloud Storage to provide scalable, secure, and redundant storage solutions. The contrast analysis system 130 and the speckle data structuring system 140 can use computational libraries and frameworks, including but not limited to MATLAB, Python with libraries such as NumPy and Pandas, or image-processing tools like ImageJ or OpenCV, to perform calculations and organize data effectively. The perfusion characterization and assessment system 150 may utilize advanced signal processing and statistical analysis software, including but not limited to MATLAB with the Signal Processing Toolbox or Python libraries like SciPy, Matplotlib, and PyWavelets, to perform Fourier transforms, time-series evaluations, and spatial analysis. Similarly, the output generation system 160 can employ visualization and reporting software, including but not limited to Tableau, D3.js, or Python libraries such as Plotly and Seaborn, to create tailored outputs, including graphs, maps, and statistical reports, for diverse end-user applications. It will also be appreciated that these software tools can operate independently or in combination, providing flexibility and enhanced functionality for processing and interpreting speckle data across clinical and research contexts.

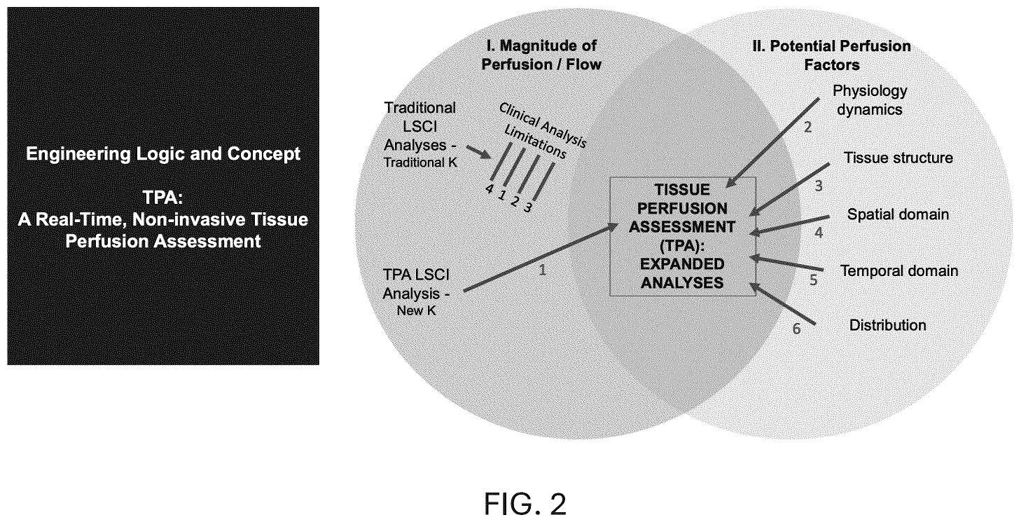

FIG. 2 illustrates an example framework for Tissue Perfusion Assessment (TPA), presented as a Venn diagram to depict the engineering principles and considerations underlying the disclosed methodologies. The framework identifies potential limitations of traditional laser speckle contrast imaging (LSCI) methods and outlines expanded analytic opportunities associated with TPA.

Component I in FIG. 2 represents traditional LSCI approaches, which can rely on speckle contrast calculations derived from spatial and/or temporal windows applied to raw speckle data. These methods may focus primarily on estimating perfusion magnitude but can encounter certain limitations, labeled as: (1) visual feedback that may lack sufficient clarity and granularity to discern subtle perfusion changes across tissues of clinical relevance; (2) challenges in interpreting results for diseased tissues due to the absence of a reference to normal perfusion in healthy tissues; (3) reliance on perfusion magnitude as the sole analytic parameter, which may not fully capture the complexity and pathophysiology of the clinical scenarios under evaluation; and (4) constraints on downstream analyses introduced by the application of spatial and/or temporal window processes. For instance, a full spatial window is without temporal context, can produce some indication of perfusion physiology but may also introduce image graininess due to significant noise, thus reducing the accuracy of perfusion estimation. A full temporal window, on the other hand, can generate averaged outputs that smooth out physiologic perfusion dynamics. With these traditional approaches each of these full windows overstate the standard deviation of intensity. This means that the magnitude estimate captures perfusion predominantly in the diastolic phase for both windows, thus underestimating actual perfusion. Also, with both, downstream domain analysis capabilities are “frozen;” with full temporal further spatial analyses are pre-defined, and with full spatial further temporal analyses are similarly constrained. Additionally, video output platforms used for magnitude evaluation in the traditional analyses may involve image processing or smoothing steps that can modify raw speckle data, further impacting the accuracy and fidelity of the analysis.

Component II identifies potential perfusion factors that may provide a more nuanced understanding of tissue perfusion when incorporated into the analysis. These factors can include: (1) physiology dynamics, (2) tissue structure, (3) spatial domain. (4) temporal domain, and (5) distribution of perfusion. Integrating these factors into the analytic process can address certain limitations associated with traditional LSCI approaches and enable expanded assessment capabilities.

The overlap of Components I and II represents an example TPA framework, which introduces an expanded approach to perfusion analysis. This framework may include the development of a new refined speckle contrast calculation (K) that can offer enhanced analytic flexibility. The new K parameter can be used to generate scalar values that support single-point time-series analyses over imaging acquisition windows, while also facilitating simultaneous evaluations of spatial and temporal domains. Unlike traditional methods, the TPA approach may reduce constraints associated with prior windowing processes, potentially enabling dynamic and physiologically relevant perfusion assessments.

FIG. 2 illustrates: (1) the potential limitations of traditional LSCI methods in supporting expanded perfusion assessments, including the constraints introduced by spatial and temporal windowing; (2) the additional factors that may be incorporated into the TPA framework to provide a more detailed evaluation, such as the inclusion of physiology dynamics, tissue structure, and distribution of perfusion; and (3) the integration of these factors through expanded analyses made possible by the new K parameter. This framework can serve as a basis for advanced tissue perfusion assessments, addressing certain challenges in existing methodologies and offering flexibility across various clinical scenarios.

### Dynamic Perfusion Evaluation and Representation Framework (DyPERF) Approach

A new approach, referred to as DyPERF, is disclosed to expand the capabilities of tissue perfusion assessment. In some embodiments, a modified method for calculating the speckle contrast parameter K is employed. Engineering principles suggest that the following requirements may be advantageous: (a) providing a direct and non-relative quantification method that reflects the underlying dynamic physiology of flow and perfusion in an intuitive manner; (b) enabling temporal and spatial scalability throughout the analytic steps; (c) ensuring applicability to raw laser speckle data acquired from hardware using sensor specifications that may include a frame rate exceeding 120 fps, suitable speckle/pixel ratios, and pixel sizes at a near-infrared (NIR) wavelength, such as 785 nm; and (d) facilitating real-time generation of analyses and visual outputs.

The DyPERF methodology can incorporate a speckle contrast calculation, denoted as K_{DyPERF}, defined as follows:

$${K}_{D y P E R F} ( i , j , n ) = \frac{{〈 {I}_{i , j , n} 〉}_{w}}{{σ ( {I}_{i , j , n} )}_{w}} \qquad ( 1 )$$

where [symbol] I_{i,j,n} [symbol] _{w} and σ(I_{i,j,n})_{w} represent the mean and standard deviation of pixel intensities across w time-frames around the n-th time-frame, respectively. Unlike conventional approaches, K DyPERF is formulated as the mean divided by the standard deviation, establishing a proportional relationship between speckle contrast and blood flow distribution. This proportionality is designed to align with and illustrate underlying dynamic physiological processes.

Once contrast images are obtained, each timeframe can be represented as a single scalar value by averaging the contrast values across the FOV, as described below:

$$\overline{DyPERF} ( n ) = \frac{1}{{n}_{r} \times {n}_{c}} {\sum }_{i = 1}^{{n}_{r}} {\sum }_{j = 1}^{{n}_{c}} {K}_{DyPERF} ( i , j , n ) \qquad ( 2 )$$

where D̅y̅P̅E̅R̅F̅(n) represents a time-series depiction of the areal average speckle contrast over the FOV. This scalar value and its corresponding time-series representation may provide insight into the magnitude of perfusion within the tissue.

### Factors Beyond Magnitude

The disclosed methods also consider factors extending beyond perfusion magnitude to enhance clinical and diagnostic applicability. These factors can include, but are not limited to, physiological dynamics, tissue structure, spatial and temporal domain scalability, and perfusion distribution, as described below:

Physiological Dynamics: Cardiovascular physiology influences normal perfusion distribution, particularly through pulsatile flow caused by cardiac cycles. This pulsatility is evident in healthy tissues and may be disrupted or absent in pathological conditions, as demonstrated in microcirculatory studies.

Tissue Structure: Tissue structure, including spatial dimensions of length (x), height (y), and depth (z), affects perfusion patterns. NIR wavelengths, such as 785 nm, can achieve penetration depths of up to 5 mm, depending on tissue type, enabling detailed assessment of vascular structures.

Spatial and Temporal Domain Analyses: Spatial resolution can range from single pixels to large arrays, while temporal analyses can vary from individual frames to extended time intervals. Combined spatial-temporal analyses may facilitate detailed evaluations across different clinical scenarios.

Perfusion Distribution in Tissue Structure (DTS): Perfusion distribution, independent of magnitude, reflects tissue-specific vascular and microvascular anatomy. Variations in distribution across normal and diseased tissues may provide additional insights into perfusion dynamics.

By addressing these considerations, the DyPERF approach enables dynamic, physiologically informed perfusion assessments with applications across various clinical and research settings.

### TPA Analysis Model

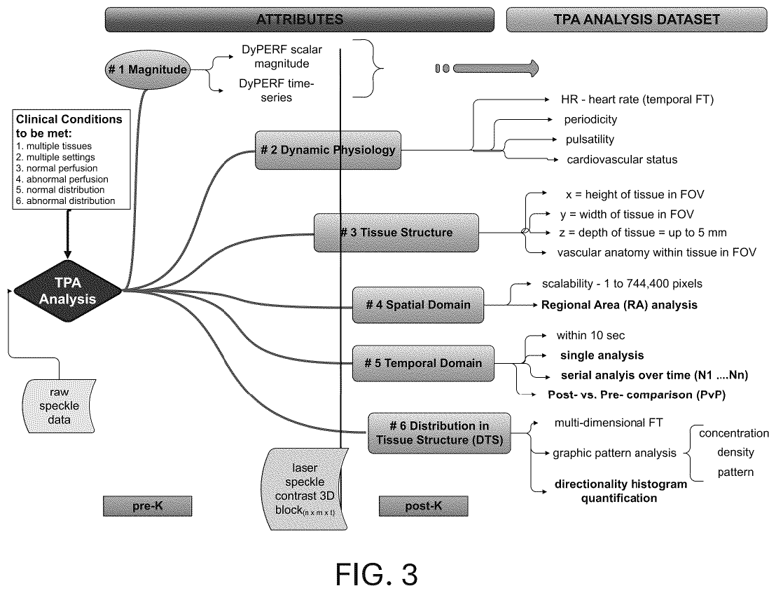

FIG. 3 illustrates an example conceptual schematic of the TPA model, which incorporates pre-K and post-K components centered around the laser speckle contrast 3D block_{(m×n×t)}. This framework integrates advanced methodologies for analyzing tissue perfusion, leveraging both raw speckle data and expanded post-K attribute analytics. The design of the TPA model is intended to meet specific clinical conditions, which are listed in the inset of FIG. 3.

The pre-K component of the model pertains to the initial calculation of laser speckle contrast (K), derived from the raw speckle data captured by a laser speckle imaging (LSI) device. The new K_{DyPERF} parameter is employed for this calculation, which allows for determination of magnitude (attribute #1) without constraining subsequent post-K analyses. The pre-K stage transforms raw speckle data into a laser speckle contrast 3D block (m×n×t), providing a foundational dataset for further analysis.

The post-K component builds on this 3D block by applying additional analytics across five validated attributes. These attributes include dynamic physiology (#2), tissue structure (#3), spatial domain (#4), temporal domain (#5), and DTS (#6). The analyses within the post-K stage are designed to address various clinical scenarios by enabling detailed assessments of tissue perfusion at multiple levels of granularity and timeframes.

Attribute #1 focuses on magnitude and is defined by the DyPERF scalar and time-series outcomes, which are the mean of all the single-point speckle contrast values from all pixels in each speckle contrast frame over time, and the unique DyPERF time-series graphic. Attributes #2 (dynamic physiology) and #3 (tissue structure) act as transitional factors, as both are embedded within the DyPERF time-series but also contribute to post-K attribute-specific analyses. Dynamic physiology captures parameters such as heart rate, pulsatility, and cardiovascular status, while tissue structure represents spatially distributed properties of tissues, including depth, vascular anatomy, and dimensions within the FOV. Tissue structure also represents transitional motion, as when the tissue structure is moving during image capture, or the camera sensor is moving relative to the stable tissue structure.

Using the post-K 3D speckle contrast block, attributes #4 (spatial domain) and #5 (temporal domain) enable scalability for analyzing perfusion data across spatial dimensions (ranging from individual pixels to the entire FOV) and temporal intervals (from single analyses to serial evaluations over time). The spatial domain also includes RA analyses, while the temporal domain supports pre- and post-intervention comparisons.

Attribute #6 (distribution in tissue structure, DTS) addresses the distribution of perfusion across tissues, incorporating multidimensional Fourier transforms (FT), graphic pattern analysis, and directional histogram quantification. This attribute provides insights into concentration, density, and patterns of perfusion that may vary between normal and diseased tissues.

The TPA analysis conceptual schematic, as shown in FIG. 3, integrates the outputs and feedback from all six attributes, generating a dataset that supports effective clinical decision-making. By allowing the weight of each attribute to be tailored to specific clinical conditions, the model provides a versatile and scalable approach for real-time tissue perfusion assessment across various settings.

The schematic further emphasizes the dual role of attributes #2 and #3 in bridging pre-K and post-K analyses, underscoring their importance in transitioning raw speckle data into clinically actionable insights. The modular nature of the TPA model allows for single or combined analyses of attributes to address complex clinical scenarios, offering a flexible and robust platform for evaluating tissue perfusion.

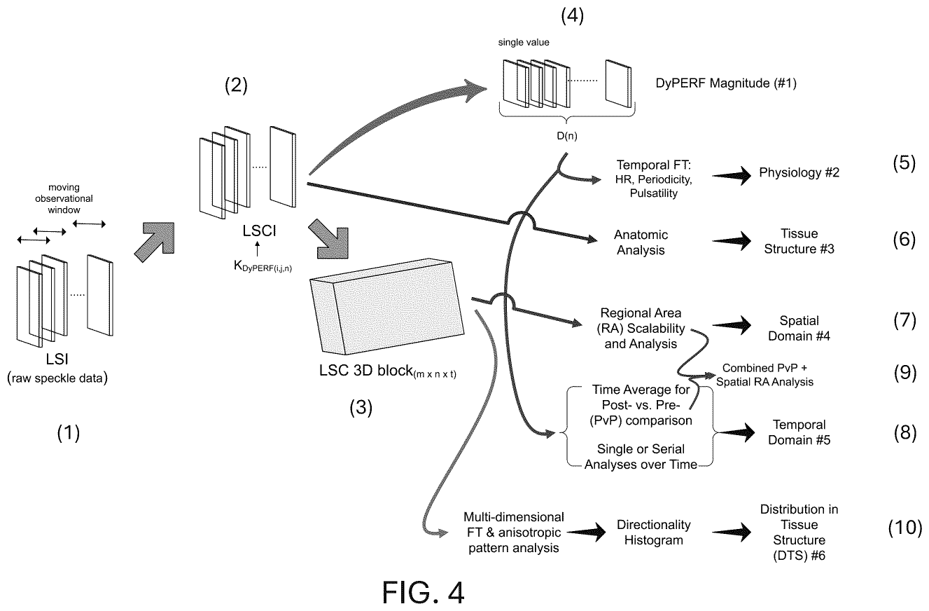

FIG. 4 illustrates an engineering graphic representation of a potential TPA model, outlining the stepwise process from initial raw speckle data to the generation of advanced perfusion assessment metrics. The figure depicts the integration of pre- and post-K_{DyPERF} stages, the incorporation of multiple attributes within the TPA model, and the resulting analytic dataset that may be produced from this approach.

The process can begin with the acquisition of raw speckle data through Laser Speckle Imaging (LSI). This data file series may be processed using a moving observational window, which can then be optimized for the clinical scenario. This optimized raw speckle dataset is not limited by spatial or temporal constraints as the substrate for calculating laser speckle contrast at each pixel by pixel using K_{DyPERF}, and the generation of a laser speckle contrast imaging (LSCI) dataset, again without spatial or temporal constraints. With this process, the laser speckle contrast 3D block (m×n×t) can be perceived, providing the foundation for potential post-K_{DyPERF} attribute-based analyses. This approach preserves full spatial and temporal resolution, enabling detailed downstream analysis.

The TPA Model, at this time, can incorporate six attributes to facilitate a detailed tissue perfusion assessment across various clinical conditions. The first attribute, magnitude, may involve the calculation of the DyPERF scalar magnitude and the generation of a DyPERF time-series. These outputs can provide both a single-value representation and a dynamic temporal visualization of perfusion, reflecting red blood cell flow and velocity in tissues. Dynamic physiology may be analyzed through temporal Fourier transform (FT) techniques, extracting physiologic parameters such as heart rate, periodicity, pulsatility, and cardiovascular dynamics. These parameters can be derived from the time-series data, capturing the dynamic nature of tissue perfusion.

Tissue structure may be integrated into the analysis by considering vascular anatomy, the spatial dimensions of tissues, including height, width, and depth within the FOV (this consideration can help align the model with the structural characteristics of healthy and diseased tissues), and translational movement of the tissue structure during imaging capture. Spatial domain analyses may provide further enhancement by supporting RA scalability and analysis. These analyses may range from individual pixels to larger regions within the FOV, enabling a granular and comprehensive assessment of perfusion. Temporal domain analyses may evaluate perfusion dynamics over time, supporting single-point evaluations and serial comparisons. This can include a combined Post-vs. Pre-(PvP) temporal format, potentially offering robust clinical insights over time intervals.

The model may also incorporate DTS through multi-dimensional Fourier transform and anisotropic pattern analysis, which can help characterize perfusion distribution patterns. The directionality histogram can quantify spatial and temporal variations, offering a detailed visualization of perfusion dynamics. Together, these analyses can form the TPA Analysis Dataset, integrating outputs from all six attributes to provide detailed insights.

The TPA Model, as illustrated, may capture quantitative and qualitative aspects of tissue perfusion. By maintaining spatial and temporal resolution and addressing certain limitations of traditional filtering methods, the model can enable physiologically relevant and real-time assessments of perfusion. The combined PVP temporal domain analysis and RA spatial analysis can enhance its potential clinical applicability, offering immediate evaluations of perfusion dynamics for a wide range of scenarios.

FIG. 4 illustrates a data flow diagram that demonstrates an example process for acquiring, processing, and/or analyzing speckle data to derive tissue perfusion characteristics and related physiological insights. This data flow diagram can correspond to the components of the system depicted in FIG. 1.

At (1), the data intake system 110 is responsible for acquiring raw speckle data using Laser Speckle Imaging (LSI). The data intake system 110 collects light intensity measurements resulting from the interaction of light with tissue across a region of interest. These measurements can include spatial variations and temporal dynamics, capturing physiological changes such as blood flow and pulsatile movements. The data intake system 110 may apply a short temporal observation window moving over the time series of each individual pixel of the entire laser speckle files for calculating speckle intensity and derivatives. This optimized raw speckle data forms the foundation for all subsequent analysis within the system.

At (2), the contrast analysis system 130 processes this optimized raw speckle data into laser speckle contrast data, by calculating K_{DyPERF}(i,j,n) at each pixel for each LSI frame. K_{DyPERF} is used to enhance the accuracy and robustness of the contrast values, to make intensity directly related to K and speckle contrast directly related to perfusion, and to permit full downstream analyses. The contrast analysis system 130 enables the transition from raw light intensity measurements to quantifiable datasets representing tissue perfusion characteristics.

At (3), the speckle data structuring system 140 organizes the laser speckle contrast data into a three-dimensional dataset referred to as the laser speckle contrast (LSC) 3D block (m×n×t). The speckle data structuring system 140 integrates spatial dimensions (X and Y coordinates) with a temporal axis (t) to form a structured dataset that allows for simultaneous spatial and temporal analysis. This dataset serves as a central repository, facilitating efficient access for downstream analytical processes. By structuring the data in this manner, the speckle data structuring system 140 supports both real-time monitoring and retrospective evaluation of tissue perfusion.

At (4), the perfusion characterization and assessment system 150 calculates the DyPERF Magnitude from the LSCI data, which represents the tissue perfusion state as a single scalar value or as a time-series curve D(n). This calculation reflects the overall perfusion magnitude characteristics of the region of interest and provides a baseline for further detailed evaluations.

At (5), the perfusion characterization and assessment system 150 performs temporal Fourier Transform analysis on the time-series curve D(n) to extract physiological parameters such as heart rate, periodicity, and pulsatility. The system isolates frequency components associated with these physiological dynamics and calculates corresponding metrics, such as beats per minute for heart rate. This analysis enables the characterization of the physiological behavior underlying tissue perfusion.

At (6), the perfusion characterization and assessment system 150 conducts anatomical analysis by examining spatial patterns within the LSC 3D block. The system evaluates vascular and microvascular structures influencing perfusion, providing insights into how tissue architecture affects blood flow. This analysis can be particularly useful in identifying anatomic structural anomalies or translational movement disruptions in perfusion.

At (7), the perfusion characterization and assessment system 150 performs regional analysis by segmenting the LSC 3D block into defined areas corresponding to specific regions of interest. The system calculates localized perfusion metrics for each region, enabling high-resolution assessments of tissue perfusion characteristics. This capability supports the identification of spatial variations, such as areas of reduced blood flow or ischemic regions.

At (8), the perfusion characterization and assessment system 150 conducts temporal comparisons, analyzing perfusion characteristics over distinct time intervals. The system can compare pre- and post-intervention datasets to assess the effects of medical treatments or procedures. By calculating time-averaged values and trends across successive temporal windows, the system provides insights into the temporal dynamics of tissue perfusion.

At (9), the perfusion characterization and assessment system 150 integrates temporal and spatial analyses to deliver a comprehensive view of perfusion dynamics. By combining time-averaged metrics with regional segmentation, the system enhances its ability to detect subtle yet clinically significant changes in tissue perfusion.

At (10), the perfusion characterization and assessment system 150 performs multi-dimensional Fourier Transform (FT) and anisotropic pattern analysis to evaluate the directional characteristics of tissue perfusion. The system generates directionality histograms to represent the alignment and distribution of blood flow patterns. These analyses quantify isotropic and anisotropic components of perfusion, providing valuable insights into structural abnormalities or directional flow disruptions.

It will be appreciated that any of the steps (1) through (10) may be omitted, occur in a different order, or be combined, as desired. For example, the order of temporal and spatial analyses may vary depending on the specific clinical or research application. Additionally, the arrows in FIG. 4 indicate the potential for feedback loops, where the outputs of one analysis can inform or refine the inputs for another. For instance, insights derived from anatomical analysis at step (6) could inform adjustments to regional segmentation at step (7), enhancing the accuracy and relevance of localized perfusion metrics. Similarly, temporal dynamics extracted at step (5) could influence how spatial analyses are conducted in step (9), enabling a more nuanced interpretation of tissue perfusion patterns.

The data flow depicted in FIG. 4 exemplifies how the components described in FIG. 1, including the data intake system 110, contrast analysis system 130, speckle data structuring system 140, and perfusion characterization and assessment system 150, operate cohesively. This integration ensures that the system can acquire, process, and analyze speckle data efficiently, delivering actionable insights into tissue perfusion for both clinical and research applications.

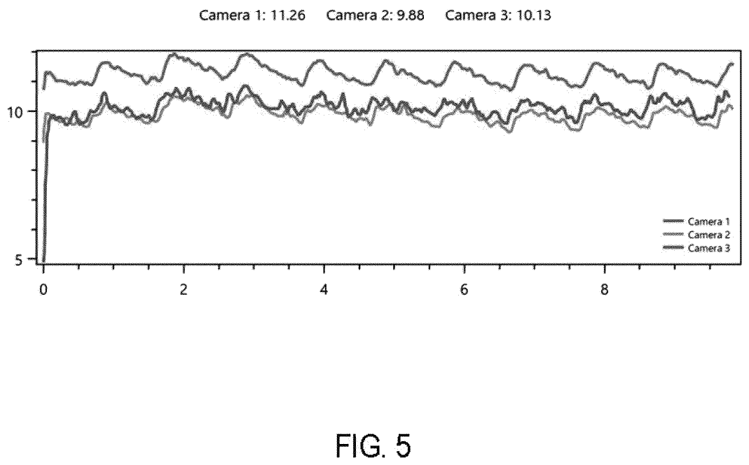

FIG. 5 illustrates an example of DyPERF time-series data that can be obtained from three different regions of the human palm, recorded concurrently using three separate cameras. The first curve represents data captured by Camera 1, which focuses on the fingertips. The second curve, captured by Camera 2, represents data from the proximal digits and palmar arch. The third curve, captured by Camera 3, corresponds to data from the base of the palm. These time-series curves can provide a dynamic measurement of perfusion calculated using the DyPERF methodology.

All three time-series curves demonstrate periodicity and pulsatility, characteristics that may correspond to physiological blood flow dynamics influenced by the cardiac cycle. These curves are derived from the processing of raw speckle data into laser speckle contrast values (K_{DyPERF}) and are directly related to red blood cell velocities within the tissues of interest. The periodic oscillations observed in the curves may reflect the natural pulsatile flow associated with the systolic and diastolic phases of the cardiovascular cycle. Over the 10-second interval depicted, the steady-state nature of the time-series can represent stable cardiovascular hemodynamics.

Above each curve, numeric values are displayed, potentially indicating the mean perfusion magnitude derived from the respective time-series data. These scalar values may summarize the overall perfusion for each region while preserving the ability to observe temporal changes.

The DyPERF methodology may provide a physiologically informed and dynamically accurate representation of tissue perfusion, while aiming to minimize spatial or temporal resolution loss. This approach can reduce reliance on processed imaging data, enhancing objectivity and minimizing the effects of translational motion. The time-series visualization may offer an immediate and intuitive depiction of perfusion dynamics, which can be valuable in applications requiring real-time physiological monitoring and perfusion assessment.

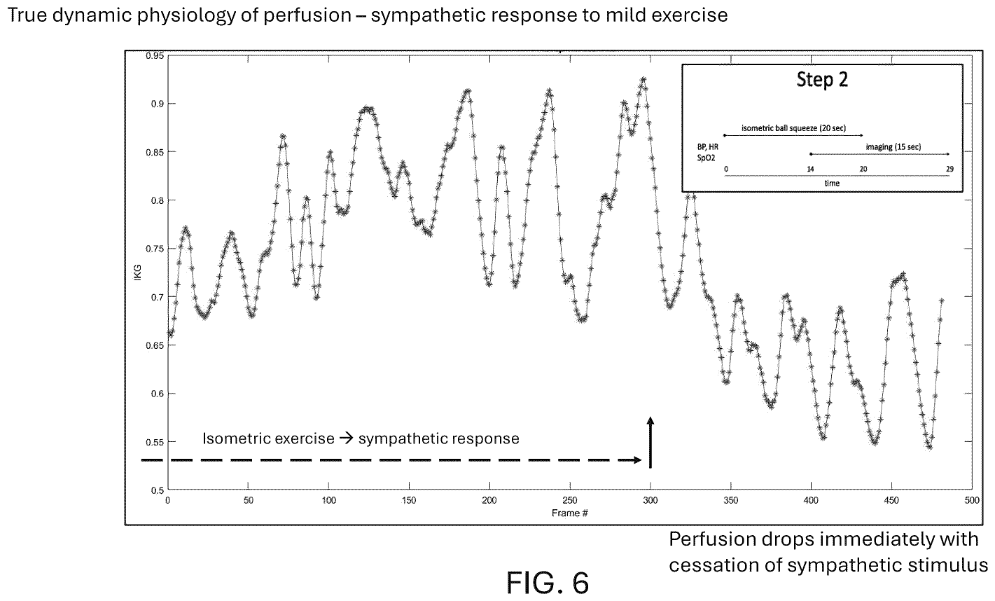

FIG. 6 illustrates an example of dynamic changes in tissue perfusion during a controlled physical maneuver, specifically an isometric ball squeeze with the L hand followed by a rest phase. These data were from a normal individual, seated during measurement of perfusion in the R palm. The inset on the left provides a timeline of the procedure, highlighting the sequence of the isometric ball squeeze (20 seconds) followed by the imaging acquisition phase (15 seconds). Parameters such as blood pressure (BP), heart rate (HR), and oxygen saturation (SpO2) are also tracked to contextualize the observed perfusion changes within physiological norms.

The graphic of the dynamic physiology of perfusion is demonstrative but not as clean as analyses using the DyPERF approach due to the spatial noise.

The time-series graph on the right depicts perfusion dynamics captured across approximately 500 frames. The x-axis represents frame numbers, while the y-axis reflects scalar perfusion quantification. Unlike FIG. 5, however, these perfusion data were calculated using a traditional analysis method using a spatio-temporal window, with calculation of K as STD/mean intensity. When perfusion is expressed as 1/K, the graphic shows increased perfusion with ball squeezing that increases sympathetic tone) vs. the subsequent return to resting perfusion following the cessation of the stimulus (indicated by the up arrow at frame 300), demonstrating a physiological change consistent with known cardiovascular responses.

This example highlights how laser speckle contrast imaging may capture steady-state perfusion characteristics alongside dynamic physiological responses to external stimuli. With this traditional analysis, however, the periodicity and pulsatility observed in the curve may in part reflect the cardiac cycle's systolic and diastolic components, but also reflects the quantum noise from the spatial component of the analysis. The physiologic relevance of the dynamic physiology of perfusion is demonstrative with this traditional analysis approach, but it is not as clean as analyses using the DyPERF approach.

The DyPERF analysis approach determines perfusion quantification directly from the raw speckle data, avoiding potential artifacts introduced by image processing or smoothing steps. This approach may preserve the objectivity and accuracy of the measurement, allowing for a graphic depiction of dynamic tissue perfusion. The periodic oscillations within the time-series may reflect normal cardiovascular physiology, whereas deviations from this pattern could indicate underlying pathologies.

Additionally, the DyPERF time-series can be utilized for advanced analytics, such as frequency analysis to estimate heart rate (HR) in beats per minute. This capability illustrates the potential versatility of the system for extracting clinically relevant parameters from perfusion data, making it potentially suitable for various diagnostic and interventional applications, including real-time surgical monitoring and transient cardiovascular assessments.

The DyPERF time-series can undergo frequency analysis to determine the dominant frequency, which corresponds to the heart rate in beats per minute. This parameter may be extracted in real-time during the production of the time-series, providing an additional measure of cardiovascular physiology. This capability enhances the system's utility by offering both perfusion dynamics and heart rate metrics simultaneously, which can be particularly valuable in clinical decision-making and monitoring applications.

The validation of the TPA model attributes was conducted using raw speckle data as input using a series of clinical studies, as outlined in Table 2, below:

TABLE 2

| Parameters | Group 1 | Group 2 | Group 3 | Group 4 |
|---|---|---|---|---|
| Device | 1 | 2 | 3 | 1 |
| Subject status | Patients | Patients | Volunteers | Patients |
| Funding | Sponsored | NIH | NIH | Sponsored |
| IRB Study | Y | Y | Y | Y |
| # Subjects | 43 | 79 | 104 | 94 |
| Disease Condition | CLTI | PAD/CLTI | none | CKD; burns; |
| | | | | GI |
| device FDA | cleared | study (fip) | study (fip) | cleared |
| Status | | | | |
| Wavelength(s) | 785; 450 | 785 | 785 | 785; 450 |
| (nm) | | | | |
| Camera Systems | 1 | 2 | 3 | 1 |
| Camera | Quest | JAI | Sony | Quest |
| frame rate (fps) | 163 | 120 | 120 | 163 |
| pixel array | 880 × 880 | 800 × 800 | 300 × 400 | 880 × 880 |
| FOV | 9 cm dia | 8 cm × 8 cm | 3 cm × 4 cm | 9 cm dia |
| speckle/pixel | 2.5 | 2.4 | 2.3 | 2.5 |
| ratio | | | | |
| working distance | 29 +/− 3 | 23 +/− 1 | 4 +/− 1 | 29 +/− 3 |
| Quantification | DyPERF | DyPERF | DyPERF | DyPERF |
| Method | | | | |
| TPA Analysis | Y | Y | Y | Y |
| Tissue SpO2 | N | Y | N | N |
| acquired | | | | |

The engineering analyses facilitated the testing and validation of candidate perfusion factors, ultimately establishing these factors as validated attributes incorporated into the TPA model. Each factor was independently assessed to confirm its unique and clinically significant contribution to the model, ensuring its applicability across diverse clinical settings.

The validation utilized raw laser speckle data acquired from three distinct LSCI devices across four clinical studies. These studies represent diverse subject groups and clinical conditions and served to evaluate and validate the candidate factors before integrating them into the TPA model. The datasets are described in Table 2, with details on device specifications, patient populations, and study conditions.

In Group 1, raw speckle data were collected using Device 1 with the specified configurations. This dataset involved patients diagnosed with CLTI as part of a study approved by WCG IRB 1303950. The device resolution met critical criteria, including a speckle size of 2.5 times the pixel size and compliance with the Nyquist equation for a 785 nm near-infrared (NIR) wavelength. The depth of penetration into palmar skin was validated as 4 mm, based on prior work with perfused optical phantoms and prior clinical studies. This resolution ensured adequate imaging of microvascular perfusion dynamics.

In Group 2, data were acquired using two synchronized camera systems operating simultaneously. This study focused on patients with Peripheral Arterial Disease (PAD) and CLTI under an NIH-funded protocol (WCG IRB 1330693). The simultaneous use of two systems allowed for comprehensive evaluation of perfusion factors in the targeted patient population. This study also simultaneously captures SpO2 from the tissues.

Group 3 involved healthy volunteers and utilized three linked and synchronized camera systems. These systems operated at a significantly reduced working distance, enabling high-resolution imaging of regional perfusion in the digits and palms. This study, conducted under WCG IRB 20214723, was funded by the NIH and provided critical data to validate the perfusion model in non-pathological conditions.

Group 4 included data collected using Device 1 across diverse surgical applications, including acute burn patients, gastrointestinal surgical patients, and chronic kidney disease (CKD) patients undergoing arteriovenous fistula procedures. The study, sponsored and approved by WCG IRB, highlighted the adaptability of the TPA model across various clinical scenarios. The data were acquired under conditions requiring imaging at different working distances and fields of view, further confirming the robustness of the model.

The devices and methodologies used across all groups adhered to rigorous specifications. The speckle-to-pixel ratio, frame rates, FOV, and working distances are detailed in Table 2. These specifications ensured compliance with optical resolution requirements, enabling high-fidelity data acquisition and analysis. The TPA model's validation leveraged the DyPERF quantification methodology to provide consistent and reliable analyses across all datasets.

All studies received approval from the respective Institutional Review Boards (IRBs) and were conducted under strict ethical guidelines. Informed consent was obtained from all participants, ensuring compliance with regulatory requirements.

The validation of the TPA model attributes confirmed its capacity to deliver dynamic, physiologically relevant, and clinically actionable insights into tissue perfusion. These results underscore the model's applicability across a wide range of patient populations, disease conditions, and clinical scenarios.

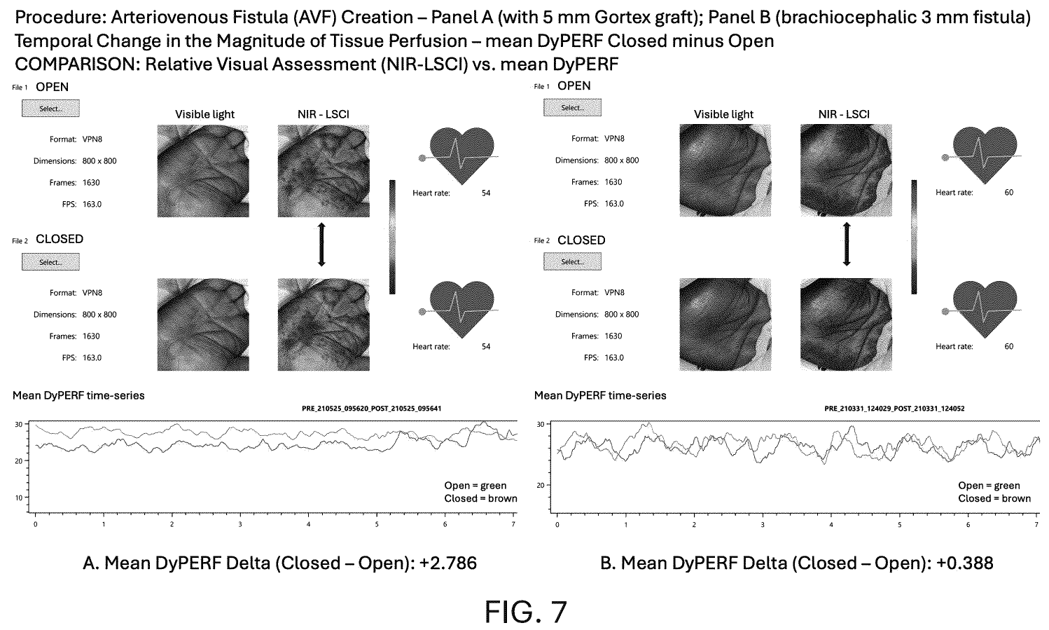

FIG. 7 illustrates a comparative analysis of perfusion magnitude determination between traditional NIR-LSCI methods and the DyPERF approach, utilizing the same raw speckle data. The data were collected during surgical creation of arterio-venous fistulas (AVFs) in the forearm in patients preparing for hemodialysis. In these acute settings, the fistula may shunt blood away from the distal palm, potentially reducing perfusion. Perfusion was assessed under two conditions: when the fistula was open, allowing blood flow, and when it was temporarily closed using a small clamp, restoring baseline perfusion.

The imaging protocol involved the sequential steps of creating the AV connection, capturing palm perfusion data with the fistula open, and repeating the capture 20-30 seconds later with the fistula closed. Data were collected using Device 1, as detailed in Table 2, under stable hemodynamic conditions. Visible light and NIR-LSCI images were obtained for both conditions, and frames were matched to the same cardiac cycle phase for consistency. Heart rate was determined from DyPERF time-series frequency analysis. Magnitude comparisons were made between visual assessments of NIR-LSCI images, referenced to a color bar, and calculated mean DyPERF delta values.

In Panel A, where a 5 mm synthetic tube graft connected the brachiocephalic artery and vein, traditional visual analysis suggested a slight increase in perfusion when the fistula was closed compared to open (arrow). However, the DyPERF analysis confirmed a significant quantitative increase, with a DyPERF delta of +2.79. In Panel B, where a direct 3 mm opening between the brachiocephalic artery and vein was created, the traditional visual analysis indicated reduced perfusion in the closed state (arrow), whereas DyPERF identified a small increase, with a DyPERF delta of +0.39. This small increase aligns with the expected physiological behavior of a direct arteriovenous fistula with a smaller opening.

The results demonstrate divergent outcomes between the two methods. In Panel A, the direction of change (increase) is consistent between visual analysis and DyPERF, but the magnitude differs substantially, with DyPERF providing a more precise and quantifiable change. In Panel B, the direction of change differs, with visual analysis indicating a decrease and DyPERF revealing a slight increase. These findings highlight the limitations of the traditional visual-based analysis as applied in real-time clinically. This traditional approach relies on subjective evaluation and lacks the clarity and granularity necessary to differentiate subtle clinical changes in perfusion.

The discrepancies between the two methods arise from the inherent limitations of the traditional NIR-LSCI approach. Temporal and spatial filtering, as well as image processing and smoothing steps, alter the raw speckle data content and impact the derived imaging results. In contrast, DyPERF directly analyzes the raw speckle data using its proprietary calculation method, avoiding data alterations and providing an objective, physiologically accurate representation of perfusion magnitude. This precision and accuracy enable DyPERF to more reliably capture subtle clinical differences in tissue perfusion, particularly in complex surgical contexts.

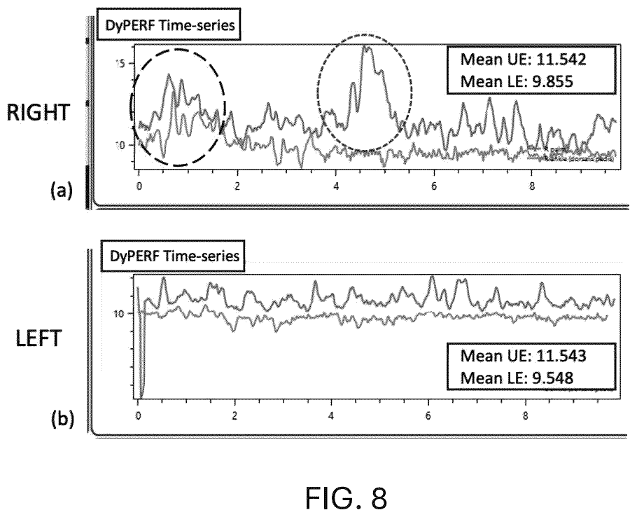

FIG. 8 illustrates the application of the DyPERF methodology to quantify tissue perfusion dynamics in the context of CLTI. Raw laser speckle data were captured synchronously and simultaneously from the upper extremities (UE) and lower extremities (LE) using identical camera systems while the patient was lying supine at rest. These time-series data reveal critical differences in perfusion between normal and diseased tissues, integrating factors related to dynamic physiology and tissue structure to provide actionable clinical insights.

As shown in FIG. 8. DyPERF scalar magnitude and single-point time-series (1,630 frames/10 seconds) were generated for the ipsilateral UE and LE. The UE time-series (depicted in green or dark curve) demonstrates normal perfusion with a consistent pulsatile flow. In contrast, the LE time-series (depicted in brown or light curve) exhibits diminished pulsatility, a hallmark of impaired but not entirely absent perfusion, indicative of severe bilateral LE CLTI. This dynamic physiology factor (Factor #2) is embedded within the time-series, offering a critical pathophysiologic marker.

Additional structural information is also evident in FIG. 8. In Panel (a), two translational motion artifacts are observed in the UE time-series. The first, marked by a black circle at 0.5-1.5 seconds, reflects residual motion from the camera system, while the second, indicated by a red circle at 4.0-5.5 seconds, corresponds to the subject moving the third digit of the right hand during imaging. These peaks, unrelated to perfusion, illustrate Factor #3 (tissue structure) and highlight the capability of the DyPERF method to identify and account for such artifacts. Should translational motion affect clinical decision-making, the imaging sequence can be repeated to ensure data validity.

Panel (b) demonstrates the normal pulsatility of the UE curve, confirming adequate perfusion. Conversely, the LE time-series curves lack such pulsatility, showing asymmetry and eventual signal loss, underscoring the severity of ischemic perfusion impairment. These findings are clinically significant as they integrate both physiological and structural insights to provide a comprehensive assessment of tissue perfusion.

FIG. 8 also incorporates the determination of heart rate (HR) via temporal Fourier transform frequency analysis of the time-series data. Pulsatility is more evident in Panel (b), where both open and closed configurations reveal physiological rhythms, though HR can still be derived in Panel (a) as well. This embedded dynamic physiology further enriches the clinical applicability of DyPERF by providing a direct correlation between tissue perfusion and cardiovascular parameters.

By integrating these elements, FIG. 8 underscores the robustness of DyPERF as a tool for assessing tissue perfusion magnitude and its determinants in both normal and pathological states, while addressing artifacts and variability inherent in imaging.

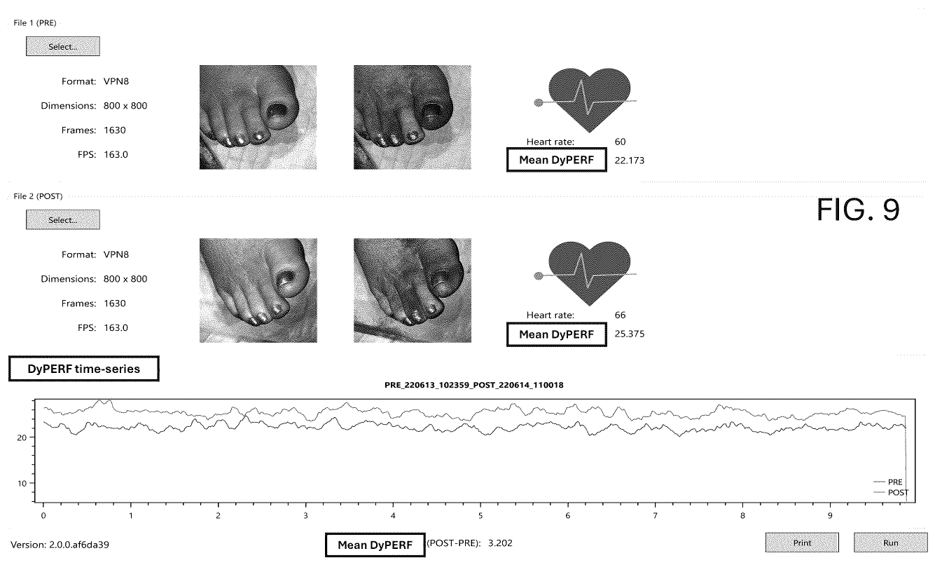

FIG. 9 presents a detailed application of the DyPERF methodology to assess changes in tissue perfusion following a peripheral arterial lower extremity intervention. The figure highlights the capability of DyPERF to objectively quantify perfusion changes, as opposed to relying on subjective visual assessments.

The DyPERF PVP analysis is depicted, where distal foot perfusion is evaluated before and after the intervention. Visible light and near-infrared (NIR) laser speckle contrast imaging (LSCI) still images are captured for reference orientation and visual quantification. As shown in the images, there is an apparent increase in red intensity (indicative of improved perfusion) in the post-intervention NIR image. However, these qualitative visual assessments lack the precision needed for clinical decision-making.

In contrast, DyPERF quantification provides a precise numerical evaluation of perfusion changes. The DyPERF time-series data indicate a measurable increase in perfusion, quantified as a delta of +3.202 between the post- and pre-intervention states. This scalar value provides an objective, actionable metric to confirm the success of the intervention.

Importantly, the heart rate (HR) during both imaging sessions remained comparable (60 bpm pre-intervention and 66 bpm post-intervention), ensuring that the observed changes in perfusion were not attributable to variations in systemic hemodynamic conditions. The robustness of DyPERF is demonstrated by its ability to isolate and quantify perfusion dynamics under consistent physiological states.

The data underscores the integration of physiological dynamics and magnitude assessment in DyPERF. The methodology resolves limitations inherent in traditional LSCI techniques, such as image smoothing and temporal filtering, which may distort raw speckle data and compromise measurement precision, particularly with small but clinically important differences in magnitude. Instead, DyPERF directly quantifies perfusion from the raw speckle data, enabling reliable and repeatable measurements unaffected by subjective interpretation.

Thus, FIG. 9 exemplifies the clinical utility of DyPERF for assessing tissue perfusion changes. Its capacity to deliver precise, objective quantification supports its application in pre- and post-intervention scenarios, contributing to evidence-based clinical decision-making in vascular and surgical procedures.

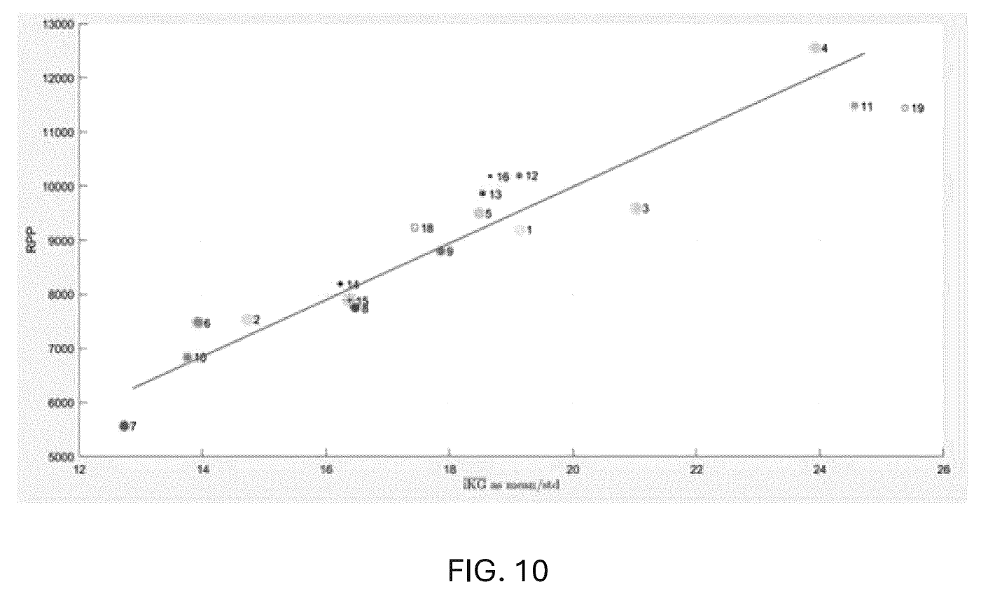

FIG. 10 illustrates the relationship between the DyPERF quantification method and traditional cardiovascular physiologic parameters, specifically the rate×pressure product (RPP), a clinical surrogate for perfusion. This correlation provides a useful framework for linking tissue perfusion metrics with standard vital signs, enhancing the clinical utility of DyPERF.

FIG. 10 represents a scatter plot where the x-axis corresponds to the scalar DyPERF values (calculated from K=mean intensity divided by standard deviation) measured from the R palm, and the y-axis depicts the RPP, calculated as the product of systolic blood pressure (SBP) and heart rate (HR). Each data point represents an individual subject, seated at rest, under controlled experimental conditions. The range of systolic blood pressures in this cohort extends from 100 mmHg to 175 mmHg.

The plot demonstrates a linear correlation between the scalar mean DyPERF values and RPP, which can be a clinical surrogate for perfusion and a link to the components of RPP. These data align along the regression line. This consistency supports the hypothesis that tissue perfusion, as quantified by DyPERF, is closely related to cardiovascular metrics indicative of systemic perfusion and workload.

Clinical studies supporting FIG. 10 indicate that DyPERF quantification (using mean Intensity/STD for K) reflects changes in perfusion dynamics influenced by physiological conditions, such as systemic vascular resistance and cardiac output that produce this range of systolic blood pressures at rest. This correlation highlights DyPERF's potential to serve as a meaningful analog for tissue perfusion, providing clinicians with metrics that align with established cardiovascular indicators.

By using the new formula for K in this analysis, the distortions often introduced by image smoothing or temporal filtering in traditional LSCI methodologies are avoided. Instead, DyPERF derives its quantification directly from raw speckle data, ensuring both accuracy and objectivity in perfusion assessment.

The findings shown in FIG. 10 demonstrate how DyPERF can complement traditional cardiovascular measurements to provide a more comprehensive understanding of perfusion dynamics. By establishing a quantitative link between DyPERF and RPP, this embodiment illustrates the potential for integrating advanced imaging analytics with traditional cardiovascular metrics, offering practical tools for real-time assessments of patient hemodynamics in a variety of clinical settings.

Spatial and Temporal Domain Analyses from the 3D Block

In some cases, the TPA model has the ability to perform analyses on the 3D laser speckle contrast block_{(m×n×t)}, where data from the block can be extracted as needed. This contrasts with the traditional laser speckle contrast 2D frames over time, to which additional spatial and temporal analyses are not possible due to the windows selected to analyze speckle contrast from the raw speckle data.

### Post-K Spatial Domain Scalability and Acuity

Engineering Analysis: assessment of perfusion in diseased tissues can require acuity and granularity to the mm level.

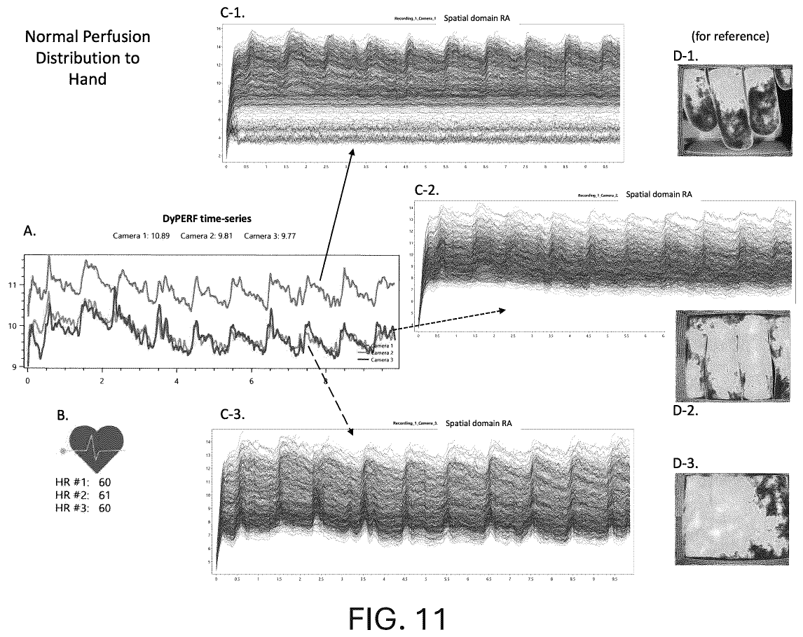

The spatial domain factor is validated in FIG. 11, which shows the link between the pre-K DyPERF magnitude time-series (Component 1 in FIG. 2) and the post-K spatial domain RA analysis (Component 2 in FIG. 2; FIGS. 3 and 4) in a unique demonstration of normal perfusion in normal tissues. These data are from TABLE 2, Group 3. All three hand segments (D-1, D-2, D-3, shown for reference only) are supplied by the same blood volume through the radial and ulnar arteries over a discrete time interval, so in theory perfusion magnitude should be nearly equivalent across all three segments. However, tissue structure can affect this, because of the clinical variability in an increased number of sensory cells in the fingertips but fewer in the skin overlying the deeper vascular plexus in the proximal digits.

FIG. 11 illustrates the Spatial Domain RA analysis of normal perfusion distribution across different hand segments, specifically the fingertips, proximal digits/palmar arch, and palm, in a normal adult volunteer. Data were acquired using three identical, fully synchronized near-infrared (NIR) reflectance laser speckle imaging (LSCI) camera systems, each with a 4 cm×5 cm FOV and a working distance of 4±1 cm. This analysis provides detailed insights into the spatial and physiological distribution of perfusion within the tissue of interest.

Panel A displays the DyPERF single-point time-series for each hand segment. The time-series data indicate that perfusion is pulsatile throughout all regions, with the scalar magnitude of perfusion being consistently highest in the fingertips (black line for Camera 1, short dotted line for Camera 2, and long dash line for Camera 3). These pulsatile patterns highlight the dynamic physiological nature of tissue perfusion in the examined regions.

Panel B presents the heart rate (HR) derived from the frequency domain analysis of the time-series data captured from each camera system. The HR values are consistent across all regions, confirming synchronized physiological activity during the imaging session.

Panel C depicts the spatial distribution of perfusion through spatial RA graphics for the fingertips (C-1), proximal digits/palmar arch (C-2), and palm (C-3). Each spatial RA graphic represents 183 time-series graphed across a 30×30 pixel RA (3×4 cm FOV). In C-1, the white background area (<8 on the ordinate) corresponds to non-perfused regions within the FOV, while perfused regions range between 8 and 16, with the largest magnitude observed in the more distal fingertip. Panels C-2 and C-3 demonstrate similar scalar magnitudes and RA ranges for the proximal digits/palmar arch and palm segments. These graphics illustrate the pulsatility of flow and the spatial distribution of perfusion over the tissue of interest, significantly augmenting the physiological and magnitude information embedded in the DyPERF time-series.

For orientation reference, NIR-LSCI images (D-1 to D-3) are provided for the corresponding segments, illustrating the visual representation of perfusion distribution in the fingertips (D-1), proximal digits/palmar arch (D-2), and palm (D-3). These reference only images were captured by the Device #3 (Table 2) using the traditional image generation algorithm in parallel. This expansion of the single-point DyPERF time-series data into this spatial domain RA analysis enhances the comprehensiveness of tissue perfusion assessment, providing both quantitative and qualitative insights.

FIG. 11 underscores the utility of spatial domain analysis in complementing the temporal and scalar information provided by DyPERF. By combining perfusion magnitude, pulsatility quantification, and regional perfusion mapping, this embodiment demonstrates a robust framework for comprehensive tissue perfusion evaluation across multiple regions of interest.

This spatial graphic display of perfusion to the hand is dramatic, in the stable physiologic symmetry of perfusion, in the tissue-structure difference influence on perfusion magnitude, and in the subtle differences between the spatial RA graphics again reflecting subtle differences in tissue structure of blood supply to the human hand. When the post-K spatial RA analysis (FIG. 4. #7) is applied to each laser speckle contrast 3D block_{(m×n×t)} corresponding to the segments and DyPERF curves, the spatial domain RA time-series (FIG. 11 C-1 to C-3) create a completely new opportunity for assessing perfusion—as an image graphic multi-point distribution of perfusion over time in the tissues of interest in the FOV.

To illustrate the potential correlation between the individual spatial RA graphic and the specific pixel area in the FOV, consider the co-location of the time-series curves with the corresponding spatial RA in the FOV. The spatial domain software generates the spatial RA time-series, a video depicting perfusion in all the scaled RAs in the FOV over time, and still image(s) from the video.

In some embodiments, once the K_{DyPERF} analysis is completed as pixel by pixel, frame by frame, and then assembled into the laser speckle contrast 3D block, the block can be spatially parsed as needed without losing any analytic resolution. For example, to quantify spatially a full FOV depicted initially by the DyPERF single-point time-series, the FOV is divided into n Regional Areas (RA), where each RA is a 2-D square, e.g., 10 mm×10 mm, 5 mm×5 mm, or 1 mm×1 mm. For example, an 8 cm×8 cm FOV, which contains 640,000 pixels, the number of pixels in each RA varies accordingly. Thus the spatial scalability can range from the entire 640,000 pixels down to the individual pixel level as the unit of DyPERF perfusion analysis over time:

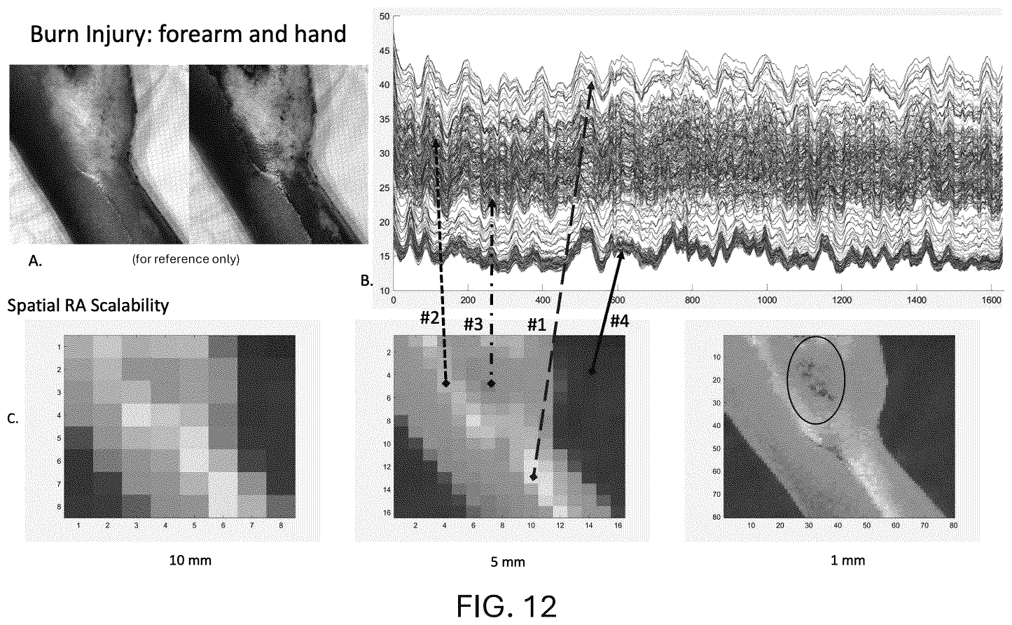

FIG. 12 demonstrates the Spatial RA analysis of acutely injured burn tissue on the medial hand and forearm. This figure highlights the ability of spatial RA techniques to achieve precise granularity and acuity for evaluating tissue perfusion in areas affected by acute injury. The raw speckle data were acquired using the Dataset 1 device in Group 4 of Table 2 and provide a detailed analysis of the spatial and physiological characteristics of the injured tissue. Perfusion is depicted by the color differentiation (yellow=high perfusion→dark blue=no perfusion).

FIG. 12 panel C illustrates a still image from the spatial RA video that is generated in the spatial RA analysis. This video is not subject to any signal processing or smoothing, but simply represents the selected number of regions and the corresponding intensity fluctuations over time. At 10 mm RA there is a gross correlation between the perfusion map and the anatomy of the burned forearm. This correlation improves as the RAs get smaller, and at 1 mm the RA perfusion is as anatomically distinct as the traditional analysis NIR-LSCI image in Panel A.

In the TPA model, these spatial RA-generated images are used for anatomic orientation of the tissues of interest. These NIR-LSCI images are used here to provide visual orientation and a qualitative representation of the perfusion distribution across the injury site, and for clarity in explaining the details of the TPA analysis approach.

Panel B shows orientation reference visible and NIR-LSCI images of the injured tissue, again generated by a traditional LSCI analysis method.

Panel B presents 256 RA time-series derived from a 5 mm RA window, offering high-resolution insights into the perfusion characteristics of the injured tissue. Four distinct regions are highlighted:
long dash arrow #1 points to an area of high perfusion.
short dash arrow #2 identifies a region of intermediate perfusion.
dot-dash arrow #3 marks the most acutely injured area with the least perfusion.

Solid black arrow #4 delineates the background area without perfusion.

These distinctions allow intuitive differentiation between varying levels of tissue perfusion and the delineation of injured versus non-injured areas, supporting informed clinical decision-making.

Panel C illustrates the scalability of RA analysis by displaying perfusion data at three different spatial resolutions: 10 mm×10 mm, 5 mm×5 mm, and 1 mm×1 mm. The color scale transitions from yellow (high perfusion) to dark blue (low or absent perfusion). At the highest resolution (1 mm×1 mm), the RA analysis image closely resembles the NIR-LSCI reference image in Panel A, confirming the precision and granularity of the technique. The black circle in the 1 mm resolution panel highlights the critical area of interest, emphasizing the importance of tailored RA window sizes based on clinical requirements.

The spatial RA graphic analysis in FIG. 12 underscores the irregular pulsatility and disorganized perfusion distribution associated with acute burn injuries. These characteristics are distinctly different from the organized patterns observed in normal tissue perfusion, as depicted in the FIG. 11 spatial RA graphics. The ability to scale the spatial resolution ensures the method can be adapted to meet specific diagnostic and clinical needs, enhancing its applicability in acute care settings.

This embodiment demonstrates the necessity of post-K spatial domain analysis to achieve a comprehensive assessment of tissue perfusion in this severely damaged tissue with greatly diminished tissue perfusion. By providing actionable insights into the severity and heterogeneity of perfusion across injured tissues, FIG. 12 highlights the robust clinical utility of the spatial RA analysis in the context of acute injury evaluation.

In some embodiments, the spatial RA scalability is also applicable when the acuity (precision) requirement is high and the granularity (discrimination size) is small, as when differentiating adjacent tissues with heterogenous perfusion. Again, FIG. 12 illustrates this post-K spatial domain analysis in an acute burn injury of the hand and forearm. As is the case in many surgical settings the juxtaposition of normal and abnormal tissue perfusion is ubiquitous, but this is particularly true in burns. This is highlighted by the black circle in the FIG. 12 1 mm image, where the darkest blue areas (no perfusion) are interspersed with lighter blue areas with impaired but greater perfusion velocities present. FIG. 12 also illustrates how familiarity with the RA graphic enables the different levels of perfusion to be readily correlated with the image data (arrows). Here, this RA spatial domain analysis contributes critical perfusion data for local tissue differentiation at the appropriate level of acuity and granularity for diseased and injured tissues, and additionally validates this attribute with important clinical data.

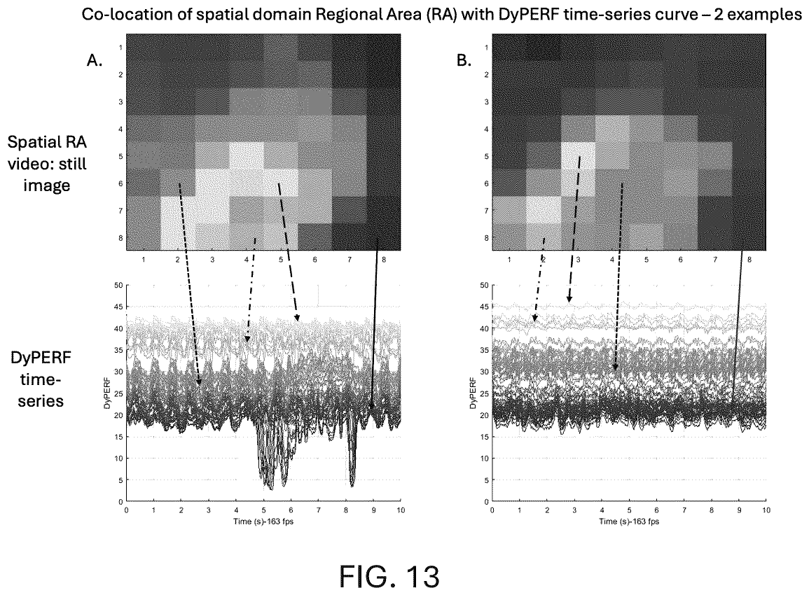

In some embodiments, the clinical precision and granularity requirements demand actual co-location of the individual spatial RA time-series with the single RA depicted in the video or still image. In both the time-series and the image the same color hue is used to highlight the co-location. In this way, the actual RA site location on the spatial RA image can be co-located with and matched in color assignment to a single time-series on the RA spatial graphic, as shown in FIG. 13.

FIG. 13 illustrates the co-location of spatial domain RAs as represented on the spatial RA image with their corresponding DyPERF time-series curves, emphasizing the integration of spatial and temporal perfusion analysis. This co-location provides a comprehensive method for correlating spatial perfusion distribution with time-series dynamics when necessary, allowing for intuitive and actionable insights into tissue perfusion patterns.

Panels A and B represent data derived from two different raw speckle data files as examples, each highlighting the spatial and temporal characteristics of perfusion within a defined tissue area. The spatial RA image in both A and B are color-coded, where brighter yellow areas correspond to regions of higher perfusion, and darker blue areas indicate regions of non-perfused or non-vital tissue.

This software sub-routine can be used when necessary to specifically co-locate the DyPERF time-series curve with the corresponding RA in the setting of juxtaposed normal and diseased tissues with normal or abnormal perfusion.

Below each spatial RA image, the corresponding DyPERF time-series curves are displayed. These curves are color-matched with their respective RA sites, providing a direct visual link between spatial perfusion intensity and temporal dynamics. The highest DyPERF time-series values align with the brightest yellow regions in the RA graphic, demonstrating consistent correlation between spatial and temporal perfusion metrics.

In Panel A, the RA graphic highlights a central region of high perfusion (bright yellow) surrounded by areas of progressively lower perfusion intensity, transitioning to non-perfused regions (dark blue) along the left and upper edges. The corresponding time-series curves reflect this gradient, with the highest values observed in the yellow regions and minimal activity in the dark blue regions.

Similarly, Panel B exhibits a different spatial distribution of perfusion, with less perfusion overall. The corresponding DyPERF time-series curves maintain alignment with the spatial data, confirming the accuracy and reliability of this co-located analysis.

This embodiment is another demonstration of the utility of determining the spatial domain analysis from DyPERF time-series data. The co-location of RA sites with their respective time-series curves enables a robust and scalable approach for assessing tissue perfusion. This integration enhances the ability to identify perfusion heterogeneity, correlate spatial perfusion patterns with dynamic physiology, and support clinical decision-making in both normal and pathophysiologic conditions. That said, in most clinical settings and with some experience in utilizing the spatial RA graphic, this correlation becomes readily apparent.

In some embodiments, combination of spatial and temporal analytics of the 3D block can be performed.

### Temporal Domain and Combined Post-K Temporal and Spatial Domain Analyses

Engineering analysis: to be applicable across a wide range of clinical conditions, post-K data analyses must be fully scalable in time and space.

To meet clinical requirements, the temporal domain requirements for this TPA analysis are variable, ranging from a single analysis to a post-intervention vs. pre-intervention (PvP) direct perfusion comparison following surgical and/or interventional therapeutic procedures (see FIGS. 3 and 4). This temporal domain framework can be used pre-K with DyPERF time-series, as shown in FIG. 7, or with post-K analyses of the laser speckle contrast block_{(m×n×t)}. These post-K domain measurement intervals can vary temporally from intraoperatively to several weeks apart; it is necessary to image the same FOV window and tissues of interest for the comparison to be an outcome quality metric.

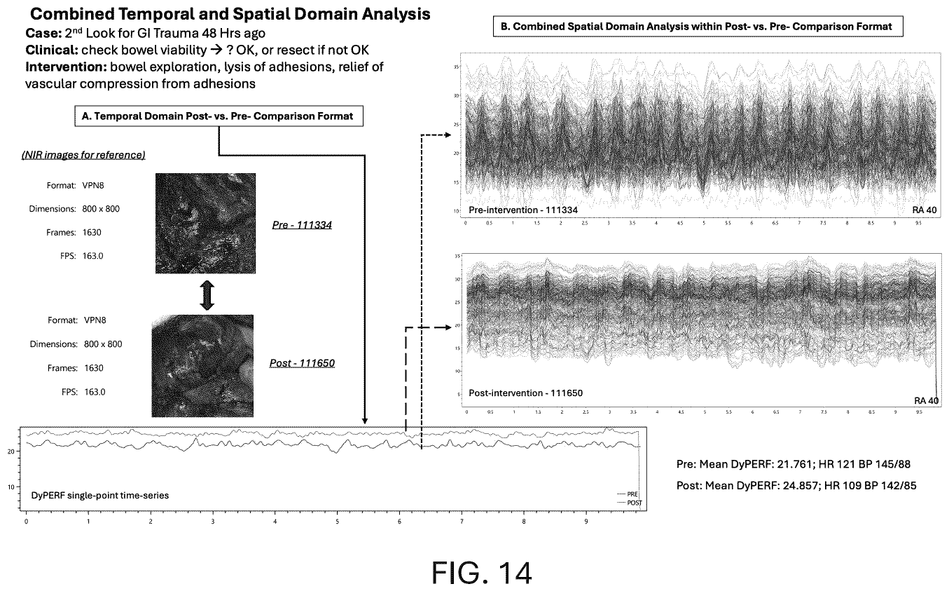

FIG. 14 demonstrates the combined temporal and spatial domain analysis of tissue perfusion using the post-K methodology in a PVP comparison format for a subject with injured bowel tissue undergoing a second-look procedure. This analysis integrates both temporal and spatial data to evaluate tissue perfusion changes following a surgical intervention, providing clinically actionable insights into tissue viability.

For reference only. FIG. 14 displays the pre-intervention and post-intervention traditional analysis method NIR-LSCI images, labeled as Pre-111334 and Post-111650. For descriptive clarity, these images provide visual context for the changes in perfusion before and after surgical lysis of adhesions and untethering of the bowel. The DyPERF single-point time-series is displayed below, capturing the temporal dynamics of tissue perfusion magnitude. The calculated mean DyPERF delta between the pre- and post-intervention states is +3.09, signifying improved perfusion magnitude following the surgical procedure. The imaging sessions occurred 3 minutes and 16 seconds apart, during which hemodynamics (BP=blood pressure, HR=heart rate) remained stable except for the increase in HR.

Panel B expands the analysis by incorporating spatial domain RA graphics into the PVP comparison format. The pre-intervention RA graphic reveals a prominent pattern of verticality in the spatial time-series curves, correlating with the faster pre-intervention heart rate (121 bpm). In contrast, the post-intervention RA graphic shows reduced verticality, increased horizontal symmetry, a greater concentration of time-series curves with higher perfusion magnitudes, and slower periodicity. These spatial and temporal changes highlight the restoration of blood flow and improved perfusion dynamics post-intervention.

The RA graphics represent 400 individual time-series curves over a 10-second interval, demonstrating the density of perfusion activity. The highest density of curves is centered around a DyPERF mean ordinate of 24 pre-intervention and 27 post-intervention. While the post-intervention RA graphic reflects improved perfusion, the graphic is not fully normalized in pattern or periodicity compared to that observed in healthy tissue, as illustrated in FIG. 11.

This combined domain analysis links the pre-K DyPERF time-series data (precise determination of magnitude) with post-K spatial domain RA analytics, offering a unique approach for assessing tissue perfusion in diseased states. The observed changes in the spatial RA graphic patterns are attributed to dynamic physiological factors (e.g., heart rate, pulsatility, periodicity) and structural attributes (e.g., anatomy of vasculature and perfusion distribution). These integrated insights support clinical decision-making by providing a scalable framework for evaluating perfusion changes in both normal and abnormal tissues.

By leveraging the spatial and temporal dimensions of perfusion data, FIG. 14 underscores the utility of DyPERF in monitoring tissue viability, particularly in complex clinical scenarios requiring intraoperative and post-surgical assessments.

### Distribution in Tissue Structure

Engineering analysis: distribution of perfusion in tissues is an aggregate of these non-magnitude attributes, can be independent of magnitude, and is critical for perfusion assessment.

The three spatial RA graphics in FIG. 11 provide a subjective assessment of normal perfusion periodicity, spatial distribution, and pattern uniformity in the distribution in the normal tissues of interest. In contrast, both FIG. 14 spatial RA graphics are abnormal as compared to FIG. 11. This difference represents a critically important further analytic opportunity, with two components: first, a single graphic of perfusion distribution can be compared to known normal perfusion in normal tissue patterns; second, the comparison of two graphics in the PVP format can illustrate the changes in the graphics distribution of perfusion, in parallel to but independent of the change in magnitude.

Since these spatial RA time-series graphics represent both a temporal and spatial distribution of perfusion, and in steady-state conditions the temporal distribution is periodic, stable, and symmetric, all the attributes validated thus far are represented in this spatial RA graphic. To further characterize this graphic, we used the three properties of distribution across space (i.e., the tissues within the FOV)—concentration, density, and pattern—to evaluate perfusion distribution as depicted in the RA time-series. Concentration is the pre-selected number of RAs (and therefore time-series curves), density is the frequency with which time-series at a similar magnitude occur in the spatial RA graphic, and pattern is the geometric organization of time-series across the space. Using these properties in a pattern analysis applied to the RA graphic provides objective quantification of perfusion distribution. This integrated analysis is termed the distribution in tissue structure (DTS).

A quantification method that is objective, simple and intuitive is required for this DTS analysis to be the final attribute in the TPA model. Pattern analysis of the RA graphic as a static image of the perfusion distribution over time provides this opportunity. A version of pattern analysis software (e.g., ImageJ, National Institutes of Health, Bethesda, MD) applied to the graphic organization (independent of the magnitude) allows for objective quantification of this DTS. In some embodiments, the integrated TPA software generates a directionality histogram of the pattern of perfusion in the tissues of interest in the FOV, starting with raw speckle data from standard LSCI hardware.

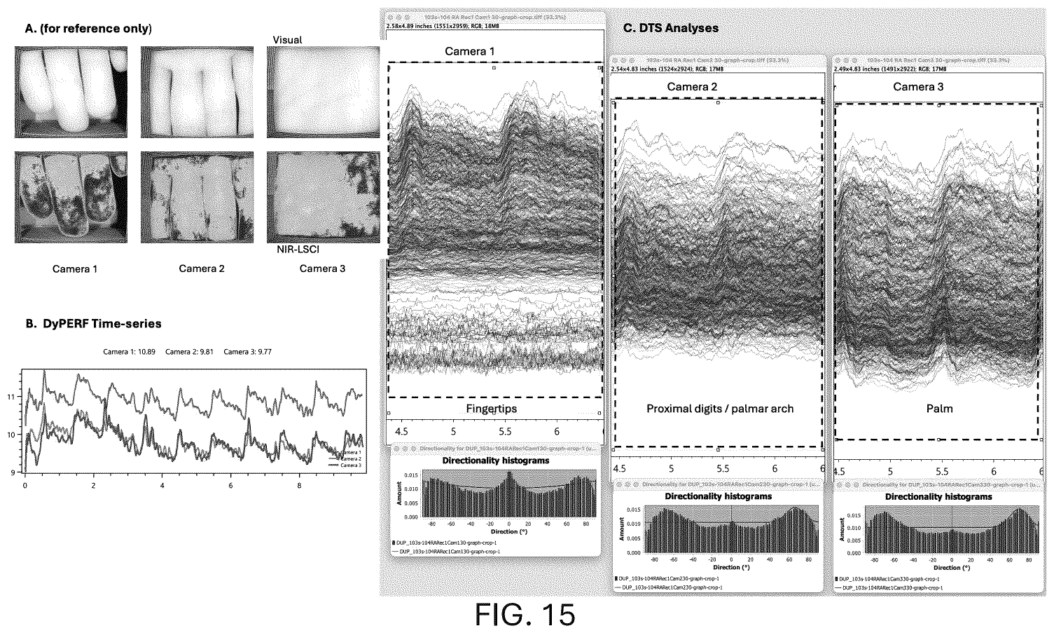

FIG. 15 demonstrates the DTS analysis as an integrated component of the TPA model. This analysis combines physiological, structural, spatial, and temporal domain attributes to characterize normal perfusion distribution within tissue structures. The DTS method provides an objective and quantifiable assessment of perfusion distribution, independent of perfusion magnitude, leveraging multi-dimensional Fourier transform and directionality histogram analyses.

Panel A provides for reference only the visual and NIR-LSCI imaging orientation of the three hand segments: fingertips (Camera 1), proximal digits/palmar arch (Camera 2), and palm (Camera 3).

Panel B depicts the DyPERF single-point time-series curves for each segment of the hand. These curves represent the temporal dynamics of perfusion across the three segments, with Camera 1 (fingertips) demonstrating the highest perfusion magnitude, followed by Cameras 2 and 3.

Panel C presents the spatial RA graphics for the three hand segments, cropped to a 1.5-second window (two cardiac cycles) from the original 10-second spatial RA time-series in FIG. 11. Using the 1.5 s window vs. the entire 10 s window results in the same DTS analysis result, but requires less computational time. This temporal segmentation also facilitates detailed DTS analyses and sub-analyses. The corresponding directionality histograms, derived through multi-dimensional Fourier transform analysis, are displayed below each RA graphic. These histograms quantify the distribution and orientation of perfusion across the spatial and temporal dimensions of the tissue of interest.

The directionality histograms exhibit the morphological characteristics of normal perfusion distribution, including tri-modal symmetry, a central peak at 0°, and anisotropic patterns with minimal skewness and kurtosis. These attributes reflect the periodicity and uniform density distribution associated with normal cardiovascular pulsatility and tissue structure. Despite differences in DyPERF magnitude and subtle structural differences among the three segments, the histograms maintain consistent morphologic features, validating the DTS method as a reliable metric for assessing normal tissue perfusion.

This analysis underscores the utility of the DTS attribute within the TPA model, enabling a comprehensive evaluation of perfusion distribution that incorporates physiology, tissue structure, and multi-domain analytics. By providing an intuitive and quantifiable metric in the directionality histogram morphology, the DTS method supports the identification of normal and pathological perfusion patterns, offering significant clinical utility for perfusion assessment across a variety of applications.

FIG. 15 exemplifies how DTS analysis can characterize perfusion distribution across different tissue segments, independent of magnitude, thereby advancing the objective quantification of tissue perfusion within the TPA framework. Camera 1=fingertips; Camera 2=proximal digits/palmar arch; Camera 3=palm of the hand.

### TPA Model Validation

Engineering Analysis: With injury, disease, or intervention perfusion magnitude may change, and perfusion distribution may change. Both need to be assessed.

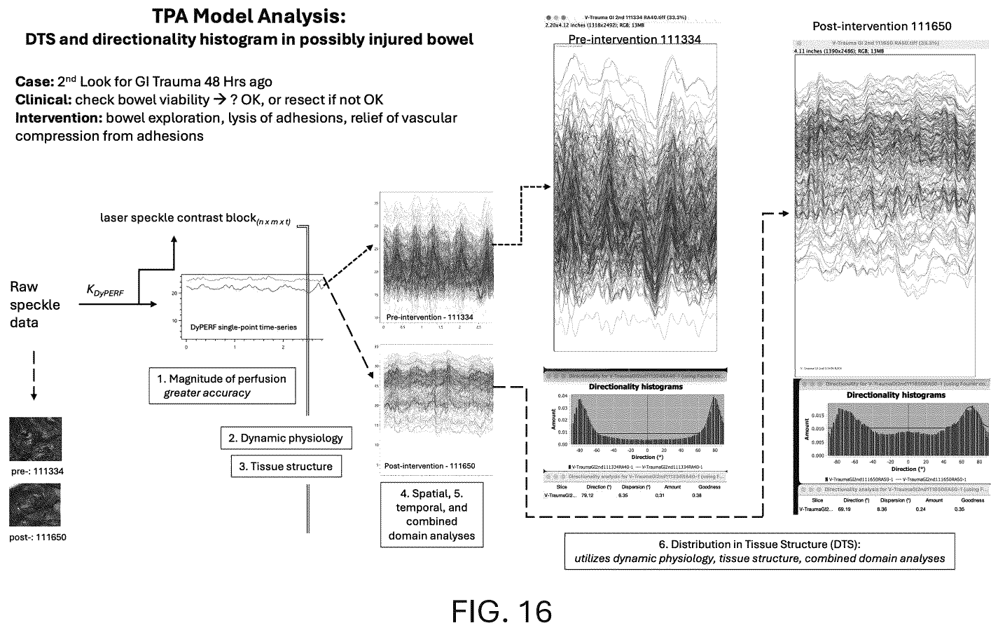

In FIG. 16, the final TPA model was tested and validated using the FIG. 14 clinical data. The FIG. 16 illustration begins with raw speckle data and generates the pre-K DyPERF single-point time series to determine the magnitude of perfusion delta (+3.09) with greater precision than existing traditional approaches. The laser speckle contrast block_{(m×n×t)} is assembled, which then allows full post-K analyses to be applied to these 3D data. The final step in the TPA model is the application of the DTS analysis, now quantified as the directionality histogram through this pattern analysis approach.

FIG. 16 illustrates the validation of the TPA model using data from the injured bowel case presented in FIG. 14. This comprehensive analysis integrates all TPA model components, including magnitude quantification, spatial and temporal domain analytics, and the DTS analysis, culminating in actionable insights for clinical decision-making.

The analysis begins with raw speckle data, which is processed to generate the pre-K DyPERF single-point time series. This provides a precise magnitude of perfusion change, with a delta of +3.09, demonstrating greater accuracy than traditional approaches. The laser speckle contrast block_{(m×n×t)} is assembled, enabling the application of post-K spatial and temporal analyses to the 3D dataset.

In the pre-K analysis, the DyPERF PVP (Post-vs.-Pre) comparison highlights relative changes in perfusion magnitude. While differences in perfusion conditions across time points may introduce variability, this stage provides a foundational reference for subsequent post-K analyses. These include advanced spatial and temporal domain evaluations, culminating in the DTS analysis.

The DTS analysis employs pattern analysis to quantify perfusion distribution in tissue structure by means of a directionality histogram. This directionality histogram reflects the relative periodicity and isotropy of the otherwise mostly anisotropic normal perfusion pattern, thus offering a direct comparator against normal perfusion patterns. For this injured bowel case, the pre-intervention directionality histogram exhibits abnormalities characteristic of isotropic perfusion patterns in severely diseased tissue. These include (i) flattening of the central trough and loss of a central peak, indicating reduced anisotropy and diminished normal periodicity and pulsatility and/or (ii) lateral peak splaying toward −90° and +90°, further reflecting isotropic and irregular flow patterns.

After the surgical intervention to restore blood flow, the post-intervention data shows marked improvement. Both the post-intervention spatial RA graphic window and histogram reflect a return toward normal anisotropy, with (i) a small emerging central peak, signifying the re-establishment of pulsatility and periodicity, and/or (ii) lateral peaks moving inward by 5-8 degrees, aligning more closely with the normal histograms depicted in FIG. 15.

This validated TPA model demonstrates its utility as a real-time clinical tool for assessing both perfusion magnitude and distribution. By combining the DyPERF delta and the directionality histogram delta, it provides a matrix of quantitative metrics that enhance clinical decision-making. The ability to track perfusion restoration and tissue recovery through both magnitude and distribution changes underscores the robustness of this model in managing complex cases involving abnormal or injured tissues.

FIG. 16 highlights how the integration of pre-K and post-K analyses within the TPA framework offers a comprehensive and scalable approach for real-time tissue perfusion assessment, supporting optimal clinical outcomes.

The model feeds back two quantitative data channels in this PVP comparison, the DyPERF delta and the directionality histogram delta. The model also feeds back two semi-quantitative channels with the full 10 s spatial RA graphic and if needed the video or still image from this spatial RA analysis. In this injured bowel setting, the pre-intervention directionality histogram includes all the pattern abnormalities seen in the pre-intervention RA graphic: the verticality of flow over time is shown in the histogram as isotropy (flattening of the central trough, loss of a central peak) and splaying out of the lateral peaks towards −90 and +90 degrees (loss of periodicity and pulsatility). This directionality histogram morphology is characteristic for the isotropic distribution pattern seen in abnormal tissues with abnormal perfusion in our studies across multiple different tissue types and clinical settings. After the successful intervention, the bowel immediately displayed restored perfusion magnitude but in addition the post-intervention histogram has recovered a major portion of the anisotropy that normal perfusion distribution should exhibit, and a small central peak is emerging. The lateral peaks have moved to toward the center by 5-8 degrees, better resembling the normal perfusion histograms in FIG. 15.

Distribution in Tissue Structure: Normal Vs. Not Normal

The “missing piece” for clinical decision-making using real-time imaging technologies is a quantitative comparable reference to “normal” tissue perfusion. In its absence, many technologies have developed analyses where quantification of magnitude has been relative within the FOV. Alternatively, attempts to weigh results against some standard magnitude for perfusion have proven unfeasible or too cumbersome for the clinical setting. In fact, there are multiple problems with both endpoints, however. Specific weighting against some standard magnitude has two problems: the standard, and clinical human variability. The standard requires some type of calibration against an established standard, and in the clinical setting this would likely require calibration before every imaging acquisition. This is incompatible with most surgical procedural workflows. Since blood flow in any tissue is fundamentally dependent on the underlying status of the cardiovascular system (e.g., BP, HR, NSR), even with such a standard cross-patient comparisons of flow would not be accurate.

In some clinical situations, the FOV contains areas of tissue perfusion that are clinically interpreted as being “normally perfused tissues.” In a relative comparison, these can be used as a “normal” reference point, with the caveat that in fact those tissues might appear normal clinically but in fact might not be normally perfused. Both NIRF and LSCI, in using visualization of a video analysis output as the basis for quantification, comparison and clinical interpretation, are subject to this caveat.

Also with these technologies, for example, clinical interpretation based on this visualization has proven to be too heterogenous across providers to be clinically useful. This heterogeneity is largely due to: 1) the difficulty in visual quantification of small differences in perfusion magnitude, and 2) the lack of a non-image “normal” reference point as part of the analysis. Providers want to know “ . . . is it normally perfused tissue, or not?” and, following an intervention, “ . . . did I make it better or not?” Without a “normal” reference point, these become entirely subjective determinations.

The algorithms for NIRF and LSCI have been tweaked over the past decades to try and address this problem, without much success. For NIRF, any such analysis remains a post-hoc process because of the requirement for dye injection and the pharmacokinetics of the dye bolus passing through the tissues of interest. For LSCI, the focus remains on determining magnitude as the only metric of perfusion. The traditional LSCI analyses can be now computed in real-time, but the output product as the imaging result is fundamentally altered from the raw speckle data by the traditional analysis windows and the smoothing image processing steps that are required to produce the video output. As demonstrated in FIGS. 7, 9, 11, 12 and 14, discrimination based on visual comparison alone of the small deltas in perfusion that are critically important clinically cannot be accurately and consistently interpreted even by clinical experts in LSCI imaging.

More recent efforts to improve precision of magnitude have attempted to remove the cardiovascular noise contribution to analyses using the speckle window for laser speckle contrast. Alternately, the temporal window has already eliminated this cardiovascular noise. From a clinical perspective, however, eliminating these physiologic components on which actual perfusion is based results in an analysis that does not accurately represent perfusion in tissues, and which cannot be used for accurate clinical decision-making.

The final clinical circumstance that makes this traditional approach largely inconsistent with accurate clinical decision-making is that most of the clinical scenarios where real-time imaging could be most effective in augmenting decision-making are in the setting of disease conditions where there are significant decreases in tissue perfusion but also significant pathophysiologic changes in the tissues themselves (e.g., diabetes, peripheral vascular disease, burns, and wounds, prolonged bowel ischemia, and tumors). There is no “normal” reference point available in the FOV, or in the tissues being evaluated.

Therefore perhaps the most important consequence of the DTS analysis is to provide this “normal” reference comparison in all the above circumstances. The DTS is an aggregation of the perfusion attribute analyses of physiology, tissue structure, spatial and temporal domains, independent of magnitude. As shown in FIG. 15, if the tissues are normal and they are normally perfused, then the DH has a characteristic morphology of a central peak with lateral peaks, as shown. This is because in normal tissues with normal perfusion there is a balance of anisotropic flow distribution with periodic isotropic perturbations due to the cardiac cycle pulsatility at a certain frequency. With abnormalities in perfusion (magnitude, distribution, or both) and in tissues (disease), this balance is disrupted, and the DH becomes much more isotropic in character (loss of central peak, flattening of the trough, pushing out of the lateral peaks), as shown in the pre-intervention directionality histogram in FIG. 16.

In some embodiments, these findings are evident when the DTS analysis encompasses the entire FOV, as shown in the examples just referenced in FIGS. 15 and 16.

In other embodiments, this DTS approach can be applied as a sub-analysis to different areas of density (frequency with which time-series at a similar magnitude occur in the RA graphic) over one or more cardiac cycles from perfusion-impaired diseased tissue, where there are multiple areas of tissue heterogeneity. The areas that are less abnormal can be distinguished from the areas that are more abnormal, both by perfusion magnitude but also with a directed DTS as well.

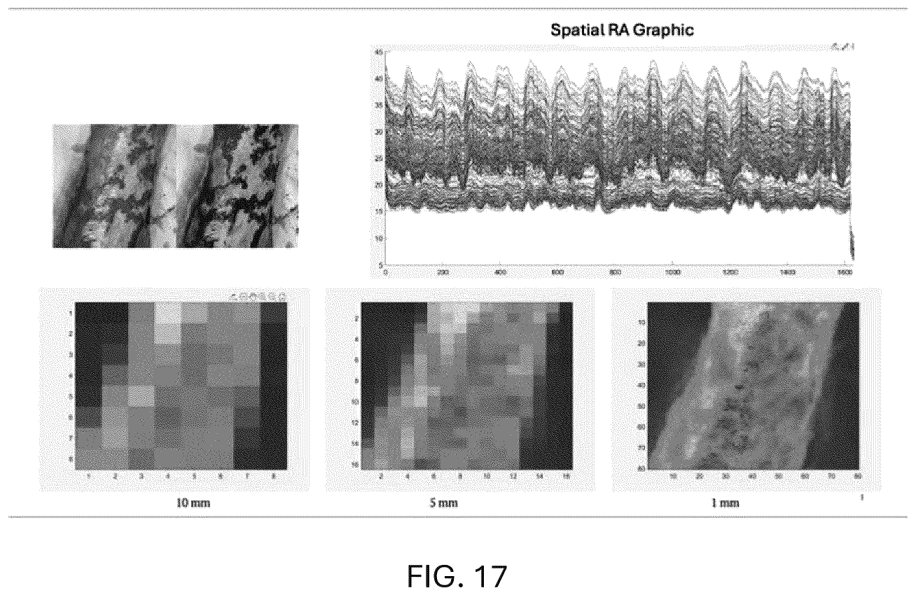

FIG. 17 illustrates the spatial RA analysis of tissue perfusion, highlighting its ability to provide quantifiable insights at varying levels of spatial detail in a different burn patient. By integrating multiple spatial RA resolutions, this analysis offers a balanced understanding of tissue perfusion patterns and their potential clinical relevance.

The upper-left panel provides for orientation only the visible light and Nir-LSCI visual outputs (a single frame from a 10-second video of the tissue being evaluated). These images show anatomical orientation, but quantifying the heterogenous characteristics of flow in the tissues based on this subjective visual representation is too complex, limiting the precision of this approach.

The lower panels display spatial RA still images derived from the same raw speckle data at three spatial RA resolutions: 10 mm×10 mm, 5 mm×5 mm, and 1 mm×1 mm. The visual color scale, ranging from deep blue (low or no perfusion) to bright yellow (higher perfusion), offers a straightforward representation of perfusion intensity. At the 10 mm resolution, broader patterns emerge, providing an overview of general perfusion trends. At the 5 mm resolution, more distinct variations in perfusion become visible, allowing for greater differentiation between areas with varying flow. The 1 mm resolution provides the most detailed depiction, closely resembling the LSCI image but supplemented by quantitative metrics that enable more precise evaluation of perfusion.

The upper-right spatial RA graphic features time-series plots for the 256 RAs within the 5 mm×5 mm resolution. These plots add a temporal dimension to the spatial analysis, showing how perfusion changes over time. Areas with lower perfusion, such as the density region between 22 and 28 on the ordinate scale demonstrate reduced pulsatility and quantitatively less perfusion magnitude, but still with perfusion present. The density area between 15 and 20 is background non-perfused areas on the L and R of the FOV, and does not represent actual perfusion. The random fluctuation in this density segment is mostly from quantum noise at the edges of the FOV and the non-viable areas. In contrast, regions with higher perfusion, such as 35-45, exhibit distinct systolic peaks and diastolic troughs, which align with more robust flow dynamics. Importantly, these subjective but accurate interpretations can be made simply by examining the spatial RA graphic and the video still images in this complex burn case. This combination of spatial and temporal data provides a more comprehensive perspective on perfusion characteristics, and in this case differentiate the special RA graphic ordinate layers as well.

This figure underscores the challenges of relying solely on visual assessment of quantified perfusion magnitude to interpret perfusion differences (FIGS. 7 and 9), as compared with assessment of the full temporal and spatial physiology of perfusion as in FIG. 17, particularly at finer levels of granularity. While the LSCI image offers a broad visual overview, the spatial RA-based analysis delivers more precise, objective and quantifiable information overall and at these subset density levels. This integration of spatial and temporal dimensions supports a more consistent and thorough understanding of perfusion.

FIG. 17 highlights the practical benefits of spatial RA analysis in offering measurable and reproducible data. This method supports the identification of regions with varying perfusion intensity, even in severely compromised tissues. By providing scalable and detailed insights, the spatial RA analysis enhances the ability to make informed decisions in clinical settings where understanding tissue perfusion is important.

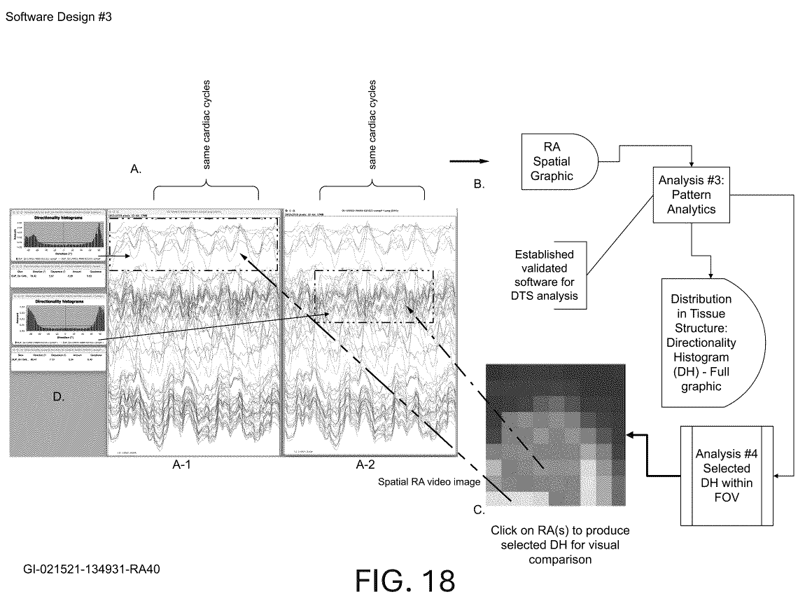

As shown in FIG. 18, the spatial RAs can have different density layers (at different levels of magnitude). But in addition, these different density layers can have different directionality histogram results for each layer as well.

FIG. 18 demonstrates this DTS pattern sub-analysis workflow, which integrates the RA spatial graphic with the DH analysis as applied to the different density layers (boxes) to generate detailed insights into perfusion distribution and magnitude differences within this single RA graphic of the entire FOV.

The schematic B begins with the spatial RA graphic derived from raw speckle data, showcasing multiple cardiac cycles. As shown in the flow diagram on the right, the DTS directionality histogram can be generated as output. But in addition, the Analysis #3: pattern analytics can be applied in Analysis #4, where a specific RA can be selected for a DTS sub-analysis, that just applies to that selected density layer corresponding to that RA in the spatial image.

In A, the same spatial RA graphic window is used for the A-1 and A-2 sub-analyses. A-1 is a density of higher perfusion, while A-2 is a density of less perfusion, as outlined by the dash-dot boxes. The corresponding DHs are generated and compared, in D. Despite originating from the same cardiac cycles, these RAs exhibit distinct DH morphologies, reflecting differences in the distribution of perfusion within the tissue structure in addition to the differences in perfusion magnitude. These differences are represented in the morphology of the directionality histogram curves and in the color variations of the spatial RA image C. The presence of unique DH sub-analysis morphologies in D confirms that differences in magnitude can be accompanied by corresponding differences in tissue distribution patterns.

The workflow includes two primary analysis stages: the full DH analysis of the RA spatial graphic and the sub-analysis of selected RA DHs within the FOV. This layered approach enables a more nuanced understanding of perfusion, allowing clinicians to pinpoint regions of interest and evaluate their perfusion characteristics in relation to surrounding tissues.

By combining spatial and directional analyses, FIG. 18 emphasizes the value of DTS pattern analysis as a tool for characterizing the spatial distribution of tissue perfusion. This approach enhances the ability to distinguish normal from abnormal perfusion, providing actionable data to support clinical decision-making. The integration of established software tools into the DTS analysis pipeline ensures consistency, accuracy, and reproducibility in these evaluations.

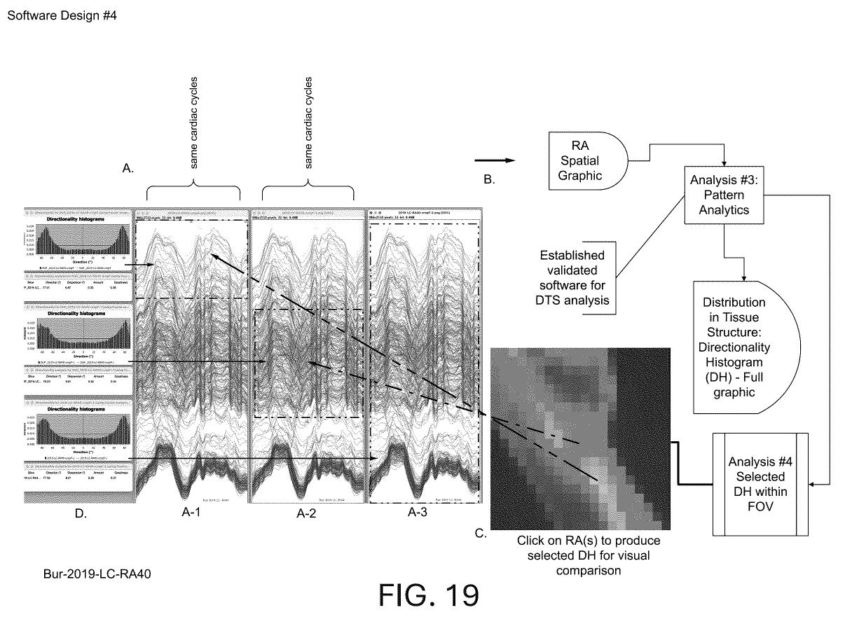

FIG. 19 presents a further application of the DTS sub-analysis, using raw speckle data from a different clinical case involving burn injury (FIG. 12). This analysis builds on the methodology demonstrated in the preceding figure, providing a comparative visualization of perfusion distribution and intensity across regions of interest (RAs) within the FOV (A-1, A-2) and the full FOV (A-3).

In this instance, the spatial RA image (c) exhibits a prevalence of no perfusion areas, represented by deep blue, low perfusion areas (green) and a more subdued presence of high-perfusion regions, as indicated by the transition from green to yellow. These differences reflect the variability in perfusion patterns associated with the severity and heterogeneity of the burn injury. The spatial RA graphic is analyzed to generate directionality histograms (DHs) for selected regions within the FOV, and the entire FOV, offering insight into the anisotropic and isotropic components of tissue perfusion.

Three DHs are generated for comparative analysis: one from the boxed density layer of a high perfusion magnitude in the first panel (A-1), another from the boxed density layer of a moderate perfusion in the middle panel (A-2), and a third encompassing the entire two-cardiac-cycle window (A-3). The spatial RA graphic subsets (in the box) exhibit similar anisotropic characteristics in A-1 and A-2, but with more isotropic components in the A-2 layer. This is seen as well in the directionality histograms in D. These subtle differences emerge in the central region around 0° and in the shape, height, and position of the lateral peaks, underscoring the nuanced changes in perfusion distribution.

The DH derived from the entire two-cycle FOV (A-3) provides a broader perspective, capturing the aggregated perfusion distribution and revealing differences compared to the region-specific DHs. This comprehensive FIG. 19 analysis highlights the utility of the DTS approach for both localized and full-FOV evaluations of tissue perfusion, enabling clinicians to assess specific areas of interest while maintaining an understanding of overall perfusion patterns.

FIG. 19 also illustrates the adaptability of the DH software analysis, emphasizing its potential for further customization and refinement as additional data are collected and new analytical techniques are developed. This iterative enhancement supports more precise and actionable assessments of tissue perfusion under varying clinical conditions, which may also be seen with additional pattern analysis metrics applied to these data.

In some additional embodiments, the DH software analysis can be further developed and customized as additional analyses and data aggregation are acquired.

### TPA Model Implementation in Different Stages of Pathophysiologic Disease

The TPA model stands out for its versatility and validated effectiveness across all tested tissue types and a wide spectrum of tissue pathophysiologic states. In some cases, a strength of the model is its treatment of magnitude and DTS as independent variables. This distinction allows the model to address clinical scenarios where the results of these analyses align harmoniously, as well as those where they diverge significantly. The flexibility to account for both outcomes is essential for accurate interpretation and application. For example, in some embodiments, the TPA model may be applied to evaluate tissues and perfusion under normal conditions, such as assessing perfusion in a skin flap before harvesting or comparing segmental perfusion to that of a normal human hand. This capability underscores the model's adaptability to both routine and complex clinical scenarios.

Engineering Analysis: In normal tissues with normal perfusion, because of the subtle differences in physiology, tissue structure, and temporal and spatial domains, both magnitude and perfusion distribution are important to tissue assessment.

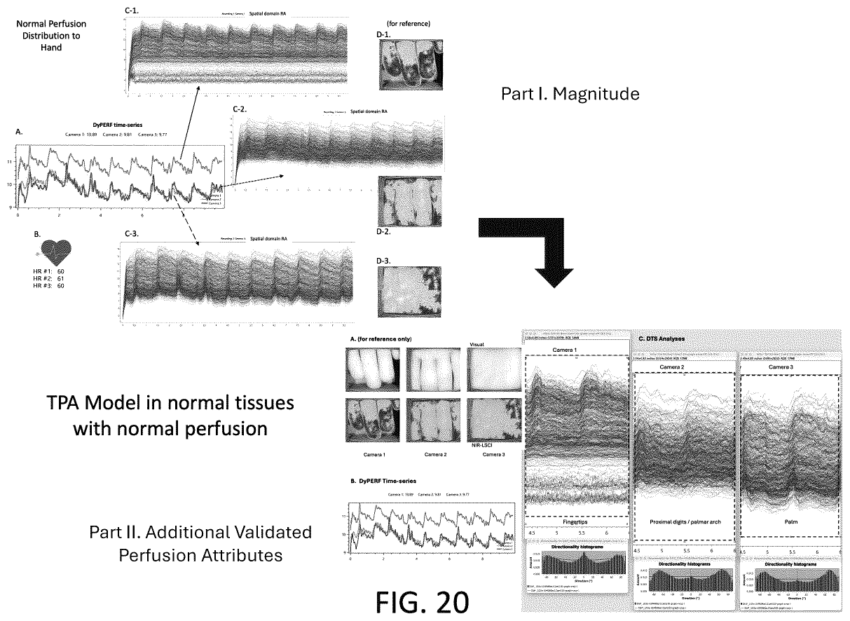

The TPA model, as applied to normal tissues under normal conditions of perfusion, offers a comprehensive framework for assessing both magnitude and distribution of perfusion. This dual assessment approach accommodates subtle variations in physiology, tissue structure, and temporal and spatial domains, which may be relevant in specific clinical settings. FIG. 20 illustrates such an application, where one or more segments of tissue are deliberately evaluated.

FIG. 20 illustrates the reproducibility and utility of the TPA model in analyzing normal tissues with normal perfusion, highlighting consistent DyPERF magnitude curves, aligned spatial RA graphics, and uniform Directionality Histograms, while also highlighting subtle variations in non-perfused regions across the evaluated tissue segments. FIG. 20 integrates data from prior analyses (e.g., FIG. 11 and FIG. 15) to exemplify the reproducibility and utility of TPA analysis in normal tissues with normal perfusion. The DyPERF curves generated by three separate camera systems demonstrate similar overall shapes, with variations observed primarily in the magnitude of the ordinate values. These variations align with expected differences in perfusion levels across sampled tissue segments.

The spatial RA spatial graphics corresponding to these evaluations further indicate a general consistency in perfusion distribution across the three areas, while also highlighting specific differences, such as the non-perfused region identified in Camera 1, Part I. This region is apparent in panel c-1 of the full spatial RA graphic below the ordinate value of 8, which corresponds to non-perfused, inanimate areas within the FOV. It is also seen in the cropped 2-cycle window (dotted window) used for the DTS analysis in Part II, C: DTS Analyses, Camera 1.

In Part II, the directionality histograms (DHs) associated with the three segments exhibit similar morphologies, reflecting uniform perfusion patterns under these normal conditions. Machine Learning analyses of these subtle histogram differences in conjunction with the differences in precise magnitude will further elucidate what these subtle differences mean and will augment the potential applicability of the TPA model to reliably assess tissue perfusion across multiple clinical scenarios. At present, the reproducibility observed in FIG. 20 underscores the potential for the TPA model to support consistent and meaningful insights into tissue health and perfusion characteristics.

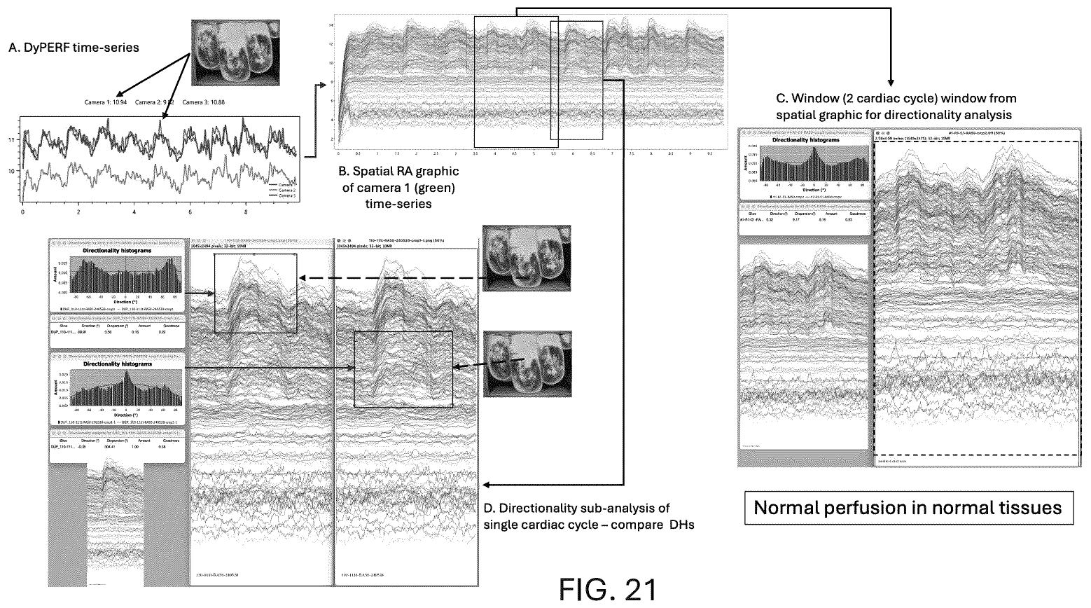

FIG. 21 illustrates the detailed application of the TPA model in analyzing normal perfusion in tissues, focusing on the distribution variability seen within a single cardiac cycle. Panel A shows a different DyPERF time-series (in green) of a fingertip segment imaged by Camera 1, with the NIR-LSCI reference image from traditional LSCI perfusion analysis for orientation only. In panel B, the spatial RA graphic of the same segment is presented, offering a view of perfusion distribution across the FOV. Two windows are selected for DTS analysis.

Panel C isolates a two-cardiac-cycle window from the spatial RA graphic for DTS analysis, resulting in a normal DH morphology reflective of standard perfusion dynamics. This provides insights into how perfusion is distributed spatially and temporally under normal physiological conditions. Panel D narrows the focus further, analyzing two different density layers from the same single cycle to compare adjacent perfusion areas of interest within the distal fingertip segment. The distal tip, characterized by higher perfusion, exhibits a normal DH, while the more proximal tissues display an anisotropic morphology, but less influenced by periodicity. This distinction aligns with physiological transitions from the sub-epithelial sensor plexus at the fingertip to deeper vascular structures in the mid-digit with likely some loss of measurable pulsatility.

This subset analysis of the DTS component of the TPA model underscores its capability to discern nuanced variations in tissue perfusion, revealing the interplay between physiology, tissue structure, and spatial-temporal dynamics. Such analyses offer an additional layer of precision and flexibility, demonstrating the model's robustness in assessing both broad and granular perfusion characteristics within normal tissues.

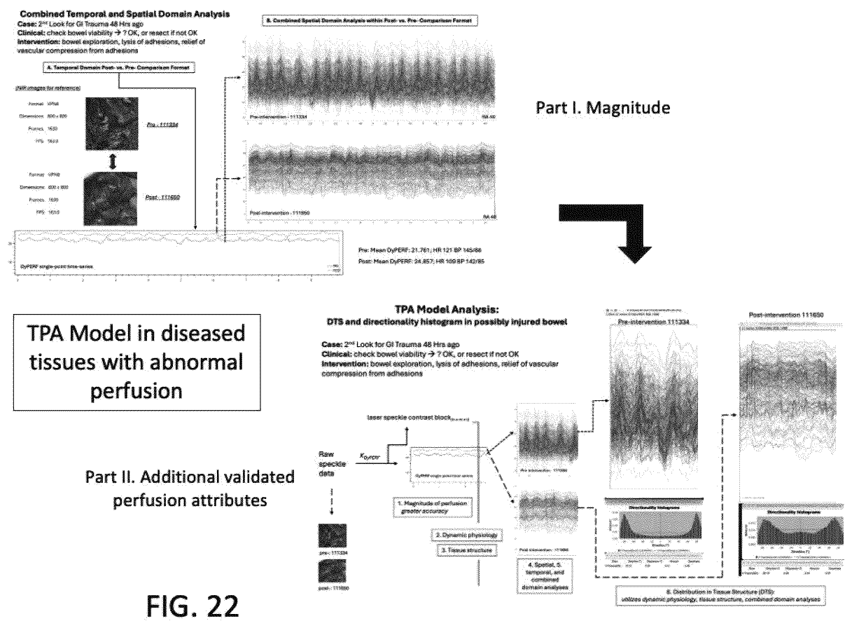

FIG. 22 illustrates the application of the TPA model to significantly diseased tissues, demonstrating its utility in integrating perfusion magnitude and the distribution in tissue structure components for a comprehensive analysis. This figure combines data from FIG. 14 and FIG. 16, providing a clear depiction of how the TPA model applies to a complex clinical scenario, such as abdominal trauma.

In Part I, the PVP DyPERF time-series data reveal a magnitude difference in perfusion to the injured bowel, with abnormal perfusion being more pronounced before the surgical intervention. However, the magnitude data alone offer limited clinical guidance because the FOV lacks a “normal” perfusion reference point—all tissues exhibit some level of abnormality. If only this PVP DyPERF time-series magnitude data were available, these magnitude insights might not meaningfully influence the surgeon's judgment because of the difficulty of putting these data into the proper clinical context. Yes, perfusion magnitude is increased, but increased from what baseline, and is the increase enough for the bowel to survive? More typically, these magnitude data alone would not surpass the specificity of traditional visual and tactile assessments made by the surgeon as standard of care.

The full TPA analysis is therefore critical in this context. The spatial RA analysis provides a more detailed understanding of the extent of perfusion and tissue-related abnormalities, offering a visual and quantitative representation of the changes brought about by the surgical intervention. Post-intervention, the spatial RA graphics highlight improvements in tissue perfusion, suggesting a positive impact of the intervention.

In Part II, the DTS analysis through the DH adds an entirely novel layer of assessment. Pre-intervention, the DH reflects significant abnormalities in perfusion distribution and tissue structure, marked by disorganized patterns and deviations from a normal DH. Post-intervention the DH improves markedly, with the DH trending toward the established more normal morphologic benchmark. Having this reference comparison to the normal DH morphology is critical for accurate perfusion assessment. Importantly, it is applicable even in the absence of normally perfused tissues within the FOV. The DH morphology and the knowledge of a normal DH morphology in normal tissue with normal perfusion provides a conceptual standard for comparison in all tissues studied thus far.

Furthermore, the DTS DH sub-analyses allow for focused evaluations of specific regions within the FOV, offering equally insightful results. These sub-analyses reveal nuanced distinctions in perfusion and tissue structure changes, enhancing the understanding of the intervention's impact.

By combining multiple layers of analysis-magnitude, spatial distribution, and DTS-FIG. 22 emphasizes the TPA model's capability to provide actionable data in complex disease scenarios. This approach ensures a more thorough and precise evaluation of tissue viability and surgical outcomes, extending beyond the limitations of traditional assessment methods.

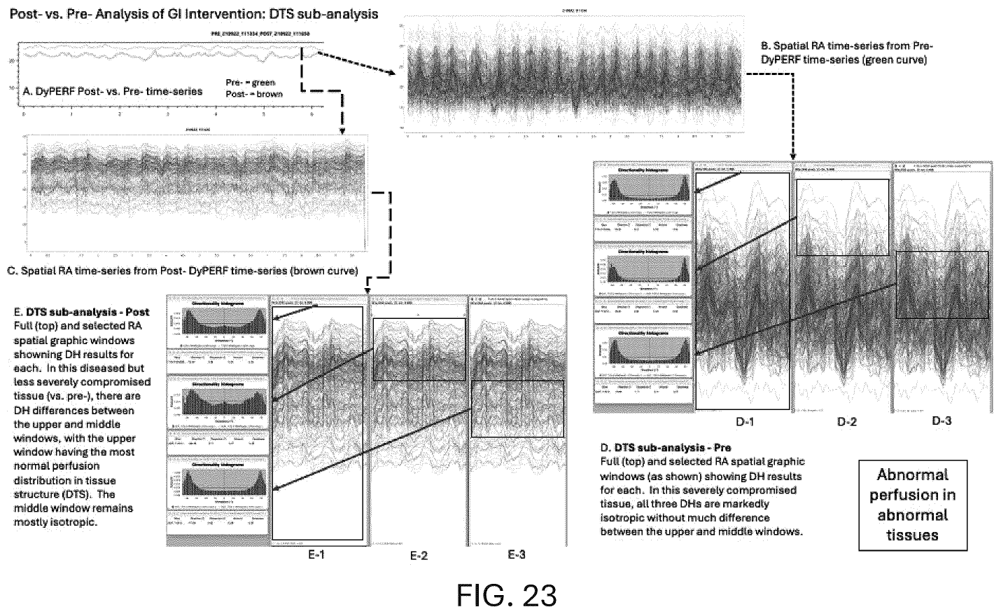

FIG. 23 illustrates the application of DTS directionality sub-analyses in tissues at two stages of disease conditions, specifically pre- and post-intervention (B and C). The pre-intervention tissues in B demonstrate a consistent and highly isotropic morphology across all three directionality histograms (D-1. D-2, D-3), which reflect these poor DTS characteristics uniformly throughout the ordinate axis range. This isotropy signifies the extent of the abnormal distribution in tissue structure before intervention.

In contrast, the post-intervention DTS sub-analyses reveal significant differentiation in DH morphologies in Panel E. The upper density layer (E-2) displays a more normal DH morphology, characterized by a reduction in isotropic characteristics and an emerging pattern consistent with improved distribution in tissue structure along with the increased magnitude. Meanwhile, the DH for the mid-range density layer (e-3) retains some isotropic features, indicating partial recovery but still reflecting the residual effects of the disease condition, as does the full DH analysis in E-1.

Overall, the comparison between pre- and post-intervention full FOV DH morphologies highlights substantial improvement in the DTS, with post-intervention tissues showing trends toward normalization in both perfusion and distribution characteristics. This is confirmed as well in the DTS sub-analyses. This FIG. 23 analysis underscores the utility of the TPA model in capturing nuanced changes in perfusion and tissue structure, even in complex and severely compromised tissues.

In some embodiments, the TPA model might be applied in clinical scenarios where magnitude and DH results turn out to be directionally convergent. In an intervention situation, this may provide important new information, allowing for a more confirmatory assessment of tissue viability in a post-vs. pre-intervention framework following the intervention.

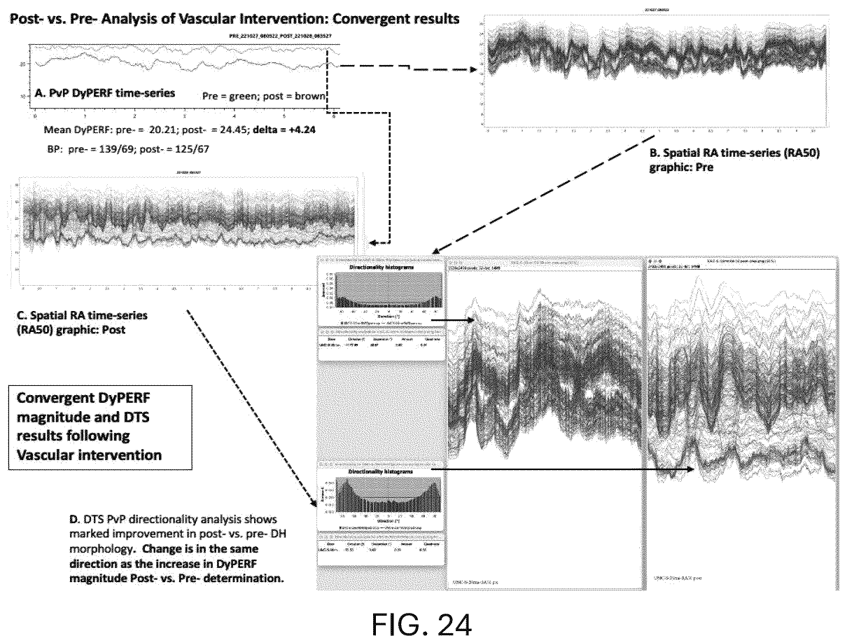

FIG. 24 demonstrates a clinical application of the TPA model, highlighting the convergence between the perfusion magnitude (DyPERF) and the DTS DH following a vascular intervention. Panel A presents the PVP DyPERF time-series before and after the intervention, with a clear improvement in the mean DyPERF value, increasing from 20.21 pre-intervention to 24.45 post-intervention, resulting in a delta of +4.24.

Panel B displays the spatial RA time-series graphic from the pre-intervention time-point, illustrating an isotropic configuration consistent with markedly abnormal perfusion in likely markedly abnormal tissue in this CLTI patient. Panel C shows the post-intervention spatial RA time-series graphic, where there is a significant shift toward a more anisotropic and normal morphology, reflecting significantly enhanced distribution in tissue structure. These visual changes documented in the two spatial RA graphics are aligned with the positive DyPERF magnitude delta, indicating a directional concordance between these two metrics.

Panel D provides a detailed DTS PVP directionality analysis, highlighting the differences in the DH configurations before and after the intervention. Pre-intervention, the DH displays strong isotropy, while post-intervention, the DH closely resembles a normal distribution, with improved peaks and reduced isotropic characteristics. The direction of change in both the DyPERF magnitude and DH morphology aligns post-intervention, reinforcing the utility of the TPA model in providing a comprehensive assessment of perfusion dynamics, and in this case the effectiveness of the intervention as measured at these time points.

In some embodiments, the TPA model may also be applied in scenarios where the DyPERF magnitude and DH results are divergent in post-vs. pre-assessments, offering additional insights into complex or heterogeneous tissue conditions.

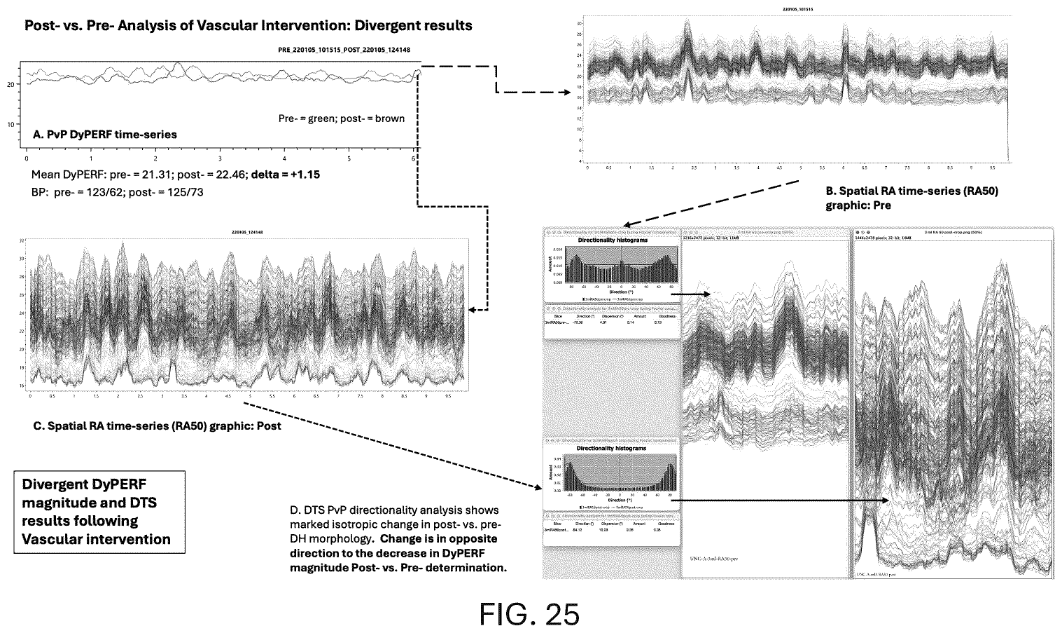

FIG. 25 provides a clinical example of divergence between the perfusion magnitude (DyPERF) and the DTS DH direction of change following a vascular intervention. Panel A displays the PVP DyPERF time-series, highlighting an increase in the mean DyPERF value from 21.31 pre-intervention to 22.46 post-intervention, resulting in a delta of +1.15. This change in magnitude suggests an overall improvement in perfusion.

Panel B presents the spatial RA time-series graphic from the pre-intervention phase, which features a morphology generally indicating a moderate compromise in perfusion, but with a high density of time-series at a high level of perfusion consistent with nearly normal perfusion distribution. However, Panel C, which shows the post-intervention spatial RA time-series graphic, reveals a striking transformation in the morphology. The post-intervention graphic is markedly more isotropic with many more levels of perfusion than pre-intervention, indicating a shift toward abnormal perfusion distribution.

Panel D quantifies the DTS PVP directionality analysis, comparing the DH configurations pre- and post-intervention. While the DyPERF magnitude demonstrates increased perfusion, the DH morphology demonstrates a contrasting trend, with an isotropic shift indicative of abnormal tissue perfusion structure and dynamics. This divergence between the DyPERF magnitude and DH results underscores the importance of integrating both metrics within the TPA model to capture the complexity of tissue perfusion and distribution dynamics. Clinically, these results could, for example, prompt additional evaluation if this divergence following the intervention was unexpected.

This example highlights the TPA model's ability to identify and interpret nuanced changes in tissue perfusion, even in scenarios where magnitude and structural distribution metrics diverge. Such analyses can offer critical insights into the underlying physiologic and structural factors affecting tissue viability and intervention outcomes.

### Software

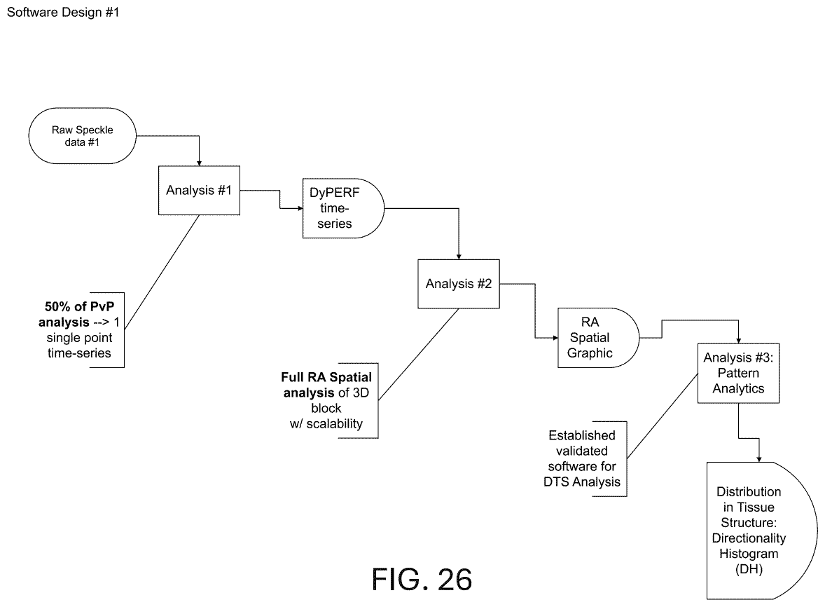

FIG. 26 illustrates an example software design for implementing the TPA model. This design can enhance precision and granularity in perfusion magnitude determination while enabling spatial scalability across parameters ranging from a single pixel to the entire FOV. It also supports the integration of spatial and temporal domain analyses for post-versus pre-interventional comparisons.

Initially developed and tested in MATLAB (R2022b, Natick, MA, USA), the design has been extended using a C++ software platform for potential clinical applications. This platform is designed to integrate attribute analyses, generate time-series data, perform post-versus pre-comparisons, and create the single or multiple spatial RA time-series graphics and video output. It can also incorporate temporal analyses and multi-dimensional Fourier transform applications. Parallel GPU processing is utilized to help reduce computational time and maintain real-time analytical capabilities.

The process begins with raw speckle data undergoing an initial analysis (Analysis #1), which can generate speckle contrast and DyPERF time-series data, contributing significantly to the post-versus pre-intervention assessment by providing single-point time-series outputs. A subsequent RA spatial analysis (Analysis #2) applies to a three-dimensional data block, offering scalability to suit various clinical needs.

The RA spatial graphic resulting from Analysis #2 is available for further pattern analysis (Analysis #3), which may include generating DTS directionality histograms (DH). Established validated software or equivalent tools can be used to support these analyses, providing insights into perfusion pattern analyses and characteristics.

This example software design illustrates how the TPA model can be implemented to address diverse analytical requirements while supporting real-time operation, from the initial capture of raw speckle data to the delivery of detailed post-analysis outputs.

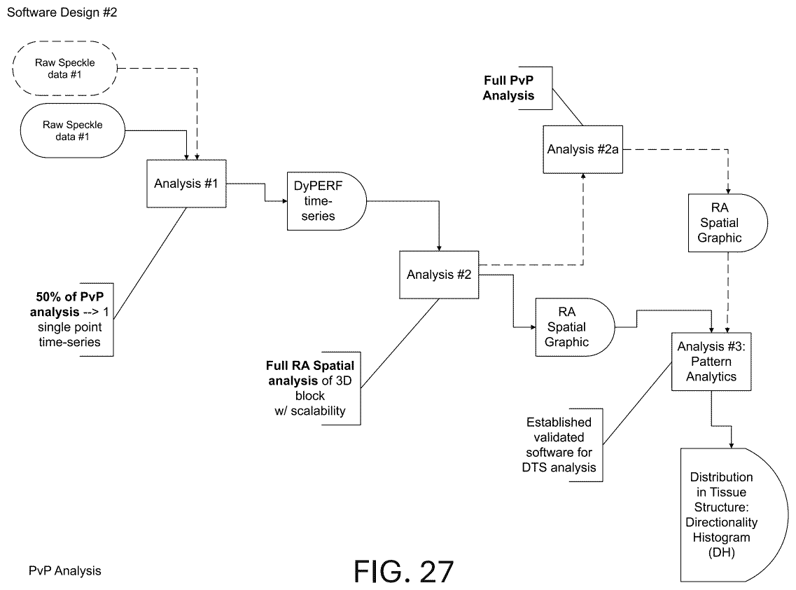

FIG. 27 illustrates an example schematic for a software design focused on post-versus pre-intervention (PvP) analysis within the TPA model. This design emphasizes flexibility and modularity, allowing for tailored analytical outputs to meet specific clinical needs. The entirely modular design of the software enables these varied approaches to be executed seamlessly within the same software package.

The process begins with raw speckle data (data #1 and #2), which is processed through an initial analysis (Analysis #1) to generate speckle contrast and DyPERF time-series data for single-point time-series outputs, representing approximately 50% of the PVP analysis. For broader analysis, a full spatial RA analysis of a three-dimensional block with scalability is conducted, offering resolution options that adapt to clinical requirements.

The DyPERF time-series data then feeds into Analysis #2, which produces the spatial RA graphic. This graphic serves as a key component for further analysis, such as pattern analytics (Analysis #3) that utilize Directionality Histograms (DH) generated through established validated software. For the full PVP analysis, there are two spatial RA graphics (Analysis #2a), each of which is subject to the pattern analysis #3. These outputs provide detailed insights into the DTS, supporting a deeper understanding of spatial and temporal perfusion dynamics.

In some embodiments, the single-point DyPERF analysis and time-series are the desired outputs, offering a streamlined approach for rapid clinical decision-making. In other embodiments, the full suite of TPA Model analytics—including DyPERF magnitude and time-series, RA spatial graphics, video, still images, and DHs—can be utilized for comprehensive assessments. For intermediate scenarios, the software can deliver outputs pre-defined for the specific clinical situation, providing a balance between simplicity and detail.

This schematic (FIG. 27) demonstrates the adaptability of the software design, ensuring that the TPA model can be applied across a wide range of clinical scenarios while providing outputs tailored to the needs of the user.

### Machine Learning/AI Concepts

Machine learning (ML) and artificial intelligence (AI) methodologies can be utilized within the systems and methods described herein to enhance the assessment and characterization of tissue perfusion and viability. These methodologies facilitate the real-time evaluation of tissue properties, particularly in clinical scenarios requiring immediate and actionable insights.

The integration of ML/AI techniques enables the dynamic characterization of tissues as “normal” or “not normal” based on quantifiable parameters of blood flow distribution and tissue oxygenation. These parameters are analyzed within the illuminated FOV and are collectively referred to as Local Tissue Viability (LTV) metrics. LTV metrics include tissue perfusion (ltvPERF) and oxygenation (ltvOXY), which are derived from the multi-dimensional datasets generated by the disclosed laser speckle imaging systems.

The ML/AI-based processes leverage the raw speckle data and corresponding attributes, including spatial, temporal, and structural metrics, to refine the classification of tissues. By analyzing these attributes across a spectrum of conditions, the system can identify physiological and pathological states ranging from intact, healthy tissues to diseased or ischemic tissues. The dynamic quantification of LTV metrics enables a more granular and precise characterization of tissue states, beyond what is achievable with traditional visual assessments.

ML/AI algorithms are further configured to address the inherent limitations of visual-based evaluations, particularly in scenarios involving subtle differences in perfusion and oxygenation. These differences, often clinically significant, may fall within a range that is not discernible by human interpretation. The disclosed ML/AI techniques transform these differences into numerical and dynamic time-series data, which are analyzed and presented to the clinician in real-time.

The disclosed systems utilize ML/AI models trained on extensive datasets of laser speckle imaging results and clinical outcomes. These models are designed to recognize patterns in the multi-dimensional data blocks (m×n×t) and extract features indicative of normal or abnormal tissue viability. The training process incorporates supervised, unsupervised, or hybrid learning approaches, ensuring adaptability to various clinical contexts and imaging conditions.

By integrating ML/AI capabilities, the systems can generate predictive insights, identify trends in tissue viability, and suggest potential interventions. These insights are based on the correlation of tissue perfusion and oxygenation metrics with cardiovascular and metabolic parameters, such as heart rate, blood pressure, and systemic oxygen levels. The AI models continuously refine their predictions through feedback loops and data aggregation, further enhancing accuracy over time.

The application of ML/AI within the described framework ensures that the analysis of tissue perfusion and viability remains consistent, objective, and reproducible. These methodologies provide augmented decision-making support, allowing clinicians to assess tissues dynamically and intuitively during clinical procedures. The immediacy and clarity of the ML/AI-generated outputs facilitate their integration into real-time diagnostic and therapeutic workflows, ensuring enhanced patient outcomes.

The disclosed inventive concepts include the application of machine learning (ML) methodologies to achieve temporal and spatial scalability in Local Tissue Viability (LTV) assessment. The goal is to enable predictive analytics for Tissue Perfusion Assessment within tissues by integrating clinical and analytical criteria into a unified framework. This integration facilitates enhanced discrimination between “Normal” and “not-Normal” tissues across diverse clinical scenarios.

Laser Speckle Contrast Imaging (LSCI) provides a foundational imaging modality by detecting the velocity of red blood cells within vessels and tissues. Each pixel in the FOV contains data on flow velocity, which is visualized through a corresponding color intensity. Temporal and spatial scalability are influenced by the frame rate and pixel-to-speckle size ratio, with implications for the accuracy and reliability of data acquisition.

### Temporal Scalability

The temporal dimension of LSCI involves capturing speckle contrast data at varying frame rates to balance between reducing visual smearing and maintaining analytical precision. High frame rates reduce speckle smearing but may lower the clarity of visual-based quantification. Conversely, lower frame rates enhance spatial analyses but can introduce inaccuracies in temporal resolution. Temporal scalability ensures that the chosen frame rate aligns with the specific clinical requirements of the procedure, enabling reliable discrimination between tissues with differing perfusion and oxygenation levels.

### Spatial Scalability

Spatial scalability pertains to the resolution of the imaging data within the FOV. In a typical FOV of 8 cm×8 cm, spatial resolution can range from the granularity of individual 1 mm² regions (comprising a 10×10 array of pixels) to the entirety of the 800×800 pixel matrix. This flexibility enables tailored assessments of perfusion across scales, from detailed evaluation of localized tissue regions to broader assessments of larger anatomical areas.

The spatial scalability must be clinically driven, and able to adapt to the different needs of specific procedures. For instance, the scalability criteria for assessing perfusion at the edges of a skin flap will differ significantly from those used to evaluate an end-segment of bowel before anastomosis. This adaptability ensures that the imaging technology provides actionable insights aligned with the procedural context.

Integration with ML/AI

ML/AI systems are employed to merge temporal and spatial scalability factors, enabling robust predictive analytics for BFD. These algorithms are trained on data derived from LSCI to identify patterns and provide enhanced discrimination between “Normal” and “not-Normal” tissues. The scalability framework supports the dynamic adjustment of analytical criteria to align with procedural variations, such as tissue type, perfusion characteristics, and clinical objectives.

ML/AI methodologies further refine the correlation between temporal and spatial data, enabling predictive models to account for variations in flow velocity, speckle smearing, and pixel intensity. By integrating these factors, the system enhances the accuracy of tissue viability assessments while accommodating the diverse demands of surgical and clinical applications.

### Clinical Relevance

The scalability of LTV assessment using the TPA model ensures that the imaging technology remains versatile and clinically useful across a wide range of scenarios. By combining temporal and spatial dimensions with ML/AI-driven analytics, the disclosed concepts provide a comprehensive framework for real-time tissue evaluation. This approach supports informed clinical decision-making by delivering precise, scalable, and context-sensitive insights into tissue perfusion and viability.

### Machine Learning: Data Sources for Analysis

The data utilized in the described Machine Learning (ML) and predictive analytics methodologies originates from the sensor(s) integrated into the multi-sensor camera system. These sensors capture the raw data that forms the foundation for all subsequent analyses and the downstream generation of datasets optimized for efficient, high-volume ML processing.

The multi-sensor system captures laser speckle contrast imaging (LSCI) data that reflects tissue perfusion and flow velocity dynamics. This raw data is processed and structured into formats suitable for temporal, spatial, and physiological analytics, enabling the integration of predictive models within the Tissue Perfusion Assessment (TPA) framework. The data's inherent granularity and fidelity provide a robust input for ML algorithms designed to enhance tissue viability assessment (LTV) and Tissue Perfusion Assessment (TPA) characterizations.

The hardware specifications required for effective LSCI data capture have been established and outlined in TABLE 2. These specifications ensure compatibility with the TPA model's analytic requirements. The hardware's operational parameters encompass key factors such as frame rate, resolution, and sensor sensitivity, ensuring accurate acquisition of data across a wide spectrum of tissue types, flow conditions, and pathophysiological states.

When the hardware criteria defined in TABLE 2 are met, the TPA model's analytics have been validated to function effectively. This validation extends across a broad range of clinical conditions, supporting real-time decision-making and predictive insights into tissue perfusion and viability. By leveraging this comprehensive dataset, ML-based algorithms can refine their predictions and continuously adapt to diverse clinical scenarios, enabling enhanced discrimination between normal and abnormal tissues.

The structured and scalable nature of these data sources ensures their applicability for both immediate analytic purposes and the iterative refinement of ML models, ultimately advancing the clinical utility of the TPA framework and its associated methodologies.

### Temporal Scalability

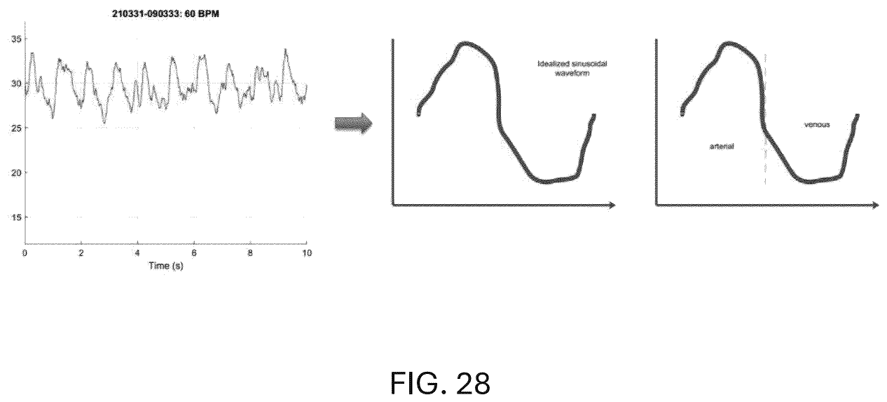

FIG. 28 illustrates the application of temporal scalability in analyzing DyPERF time-series data, highlighting the transformation of a 10-second interval of raw speckle-derived data into an idealized sinusoidal waveform that retains critical physiological and analytical features. The left panel depicts the original time-series data, characterized by periodic oscillations corresponding to individual cardiac cycles. In steady-state conditions, these cycles exhibit significant redundancy, as they represent similar patterns of systolic peaks and diastolic troughs. The middle panel demonstrates the generation of an idealized sinusoidal waveform from the raw data using mathematical transformations that incorporate frequency, offset, and amplitude. This condensed waveform encapsulates the essential characteristics of the time-series while minimizing the noise inherent in the original data. The right panel further illustrates the decomposition of this idealized waveform into arterial and venous components, allowing for a detailed analysis of the distinct contributions to tissue perfusion dynamics.

This temporal reduction process achieves several important objectives. By compressing the redundant time-series into a representative waveform, it significantly reduces the data size, enabling efficient storage and processing. For instance, the resulting idealized sine wave can occupy as little as 1 kilobyte, enhancing computational and analytical scalability. Additionally, this approach facilitates the generation of simulated time-lapse imaging sequences that reflect perfusion dynamics within the FOV, allowing for intuitive visualization of changes over time at various spatial granularities. The ability to separate arterial and venous flow components further enriches the analysis, providing nuanced insights into the mechanisms underlying tissue perfusion. By reducing the influence of noise, the temporal reduction process also ensures that the waveform accurately represents the physiological signal, even if the idealized shape deviates slightly from a pure sinusoid to forms such as sawtooth curves, depending on the signal characteristics.

This methodology is adaptable across multiple spatial granularities within the FOV, ranging from individual pixels to larger RAs. Idealized waveforms can be generated for each RA, allowing for precise and scalable analysis tailored to specific clinical and research needs. The approach demonstrated in FIG. 28 underscores the utility of temporal scalability in Machine Learning workflows, enabling advanced predictive modeling and efficient data handling within the Tissue Perfusion Assessment framework. By preserving the essential dynamics of tissue perfusion while reducing data complexity, this innovation significantly enhances the efficiency and applicability of the analytics in diverse clinical scenarios.

### Relative Quantification of Perfusion Using the Idealized Sinusoidal Curve

The relative quantification of perfusion using the idealized sinusoidal waveform is based on two foundational premises: (1) that there are no significant differences between individual cardiac cycles within a steady-state 10-second window, and (2) that the precise shape of the sinusoidal waveform is not critical to the analysis. These premises enable a significant reduction in data representation by characterizing the sinusoidal waveform through its peak and minimum values. These values can be stored as two floating-point numbers, requiring only 8 bytes on a 32-bit system or 16 bytes on a 64-bit system, resulting in a substantial decrease in data storage and processing requirements without compromising analytical accuracy.

To facilitate visualization and relative quantification, a 5-point color scale is employed to represent perfusion levels. The color scale includes red (R), orange (O), yellow (Y), green (G), and blue (B), corresponding to various perfusion intensities during the cardiac cycle. Relative perfusion is quantified by mapping the transition from the peak to the minimum value of the sinusoidal waveform to one of 15 possible color transition patterns. These transitions are defined as follows: red at the peak can transition to red, orange, yellow, green, or blue (five possibilities); orange at the peak can transition to orange, yellow, green, or blue (four possibilities); yellow at the peak can transition to yellow, green, or blue (three possibilities); green at the peak can transition to green or blue (two possibilities); and blue at the peak can transition to blue (one possibility). These transitions cumulatively form a 15-point scale.

Each of the 15 possible transition patterns is represented by a single byte of data, further optimizing storage and processing efficiency. If greater resolution is required, the 5-point color scale can be expanded to finer granularities, such as a 20-point scale, while still maintaining efficient data representation within a single byte. For significantly higher granularities, data storage may increase but will remain within the bounds of 8 to 16 bytes per unit within the field of view.

This method provides a highly efficient framework for visualizing and quantifying perfusion dynamics using minimal data resources. The integration of this approach into the field of view analysis ensures that perfusion behaviors can be represented compactly and accurately, facilitating real-time clinical decision-making while remaining compatible with existing hardware and software systems.

### Spatial Scalability

The disclosed system provides a framework for spatial scalability in tissue perfusion and flow analysis, leveraging sensor metadata to enable dynamic adaptability across varying levels of granularity. The system can process the FOV, assumed to be 8 cm×8 cm, consisting of an 800×800 pixel array, or approximately 640,000 pixels per frame. Each individual pixel contains unique DyPERF time-series data, which represent the instantaneous changes in flow and perfusion intensity over time. By comparing DyPERF time-series data for adjacent pixels, the system can differentiate flow and perfusion characteristics with unparalleled granularity. This capability establishes the foundation for generating quantitative datasets optimized for machine learning (ML) and artificial intelligence (AI) analytics. The sensor metadata approach, as hypothesized, provides an optimal data source for such ML applications.

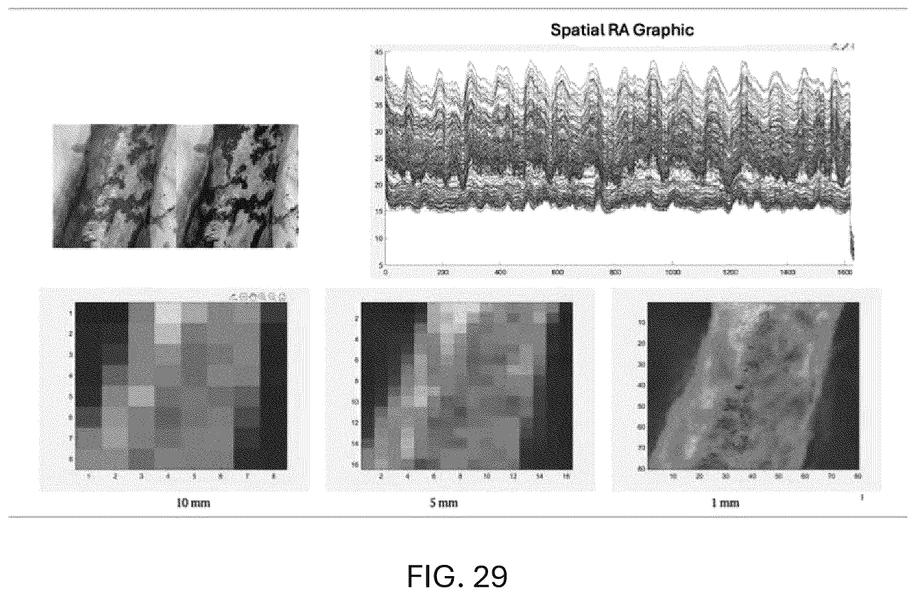

FIG. 29 illustrates the implementation of spatial scalability in tissue perfusion assessment. The upper left panel of FIG. 29 displays a laser speckle contrast imaging (LSCI) visualization, showing a visible light image on the left and the corresponding LSCI image of flow and perfusion on the right. This single frame from a 10-second video provides an overview of perfusion distribution. The lower panels depict the spatial scalability of RA granularity, with frames dividing the FOV into RA grids of 10 mm×10 mm (left), 5 mm×5 mm (middle), and 1 mm×1 mm (right). A consistent color scale, ranging from deep blue (no perfusion) to bright yellow (high perfusion), is applied across all representations, allowing immediate visual quantification of perfusion intensity differences. The spatial RA graphic shows a time-series plot for 256 RAs derived from the 5 mm×5 mm RA grid, highlighting areas of low perfusion (e.g., RAs 15-20) with minimal pulsatility versus high perfusion areas (e.g., RAs 35-45) with clear systolic peaks and diastolic troughs. The distinct granularity levels illustrate how spatial scaling enhances the differentiation of perfusion patterns beyond the limitations of visual assessment alone.

Spatial scalability considerations can include: the definition of RA granularity and data upload size optimization. Granularity, interpreted as the sharpness or keenness necessary for Local Tissue Viability (LTV) assessment, can range from individual pixels to the entire FOV. For example, a 1 mm² RA, comprising 10×10 pixels, divides the FOV into 6,400 RAs, while a 2 mm² RA, comprising 20×20 pixels, reduces the RA count to 1,600. The starting level of spatial granularity is typically 1 mm², which is below the human visual detection threshold and clinical relevance for differentiating “Normal” from “not Normal” tissues. The determination of optimal RA granularity is refined through ML algorithms applied to diverse tissue types and clinical procedures.

To address the computational and storage demands of processing high-resolution sensor metadata, the system incorporates a method for data size reduction. Each DyPERF time-series generates approximately 40-50 KB of data. For 640,000 pixels, this results in a file size of approximately 3 GB per 10-second video episode. By applying RA granularity at 1 mm² (6,400 RAs), the data size is reduced to approximately 320 MB per episode. At 2 mm² (1,600 RAs), the data size is further reduced to 80 MB. A more significant reduction is achieved by replacing individual time-series data with idealized sinusoidal curves representing the RA's perfusion behavior. This approach reduces the data size to 6.4 KB per episode for 1 mm² granularity and 1.6 KB per episode for 2 mm² granularity.

To enable these advanced spatial scalability capabilities, the system employs dedicated software for real-time processing and ML dataset preparation. The capturing device collects a 10-second sensor metadata file and generates DyPERF time-series data, which are subsequently processed to create idealized sinusoidal curves for each RA. These curves are further enhanced with color-coded patterns representing perfusion dynamics. The resulting ML-ready dataset, optimized for both spatial and temporal scalability, facilitates efficient upload and downstream predictive analytics.

This spatial scalability framework ensures applicability across a wide range of imaging data, tissue types, and surgical procedures, with FIG. 29 illustrating its versatility in providing both detailed visualizations and quantitative insights into tissue perfusion. The system's ability to scale granularity dynamically supports precision medicine and enhances ML-driven clinical decision-making.

The inventive concepts disclosed herein can incorporate multiple steps and considerations for software development aimed at testing, verifying, and validating the potential of sensor metadata as a high-probability solution for narrow AI (ANI) machine learning applications. Based on discussions with ML/AI experts, these steps can refine and implement the hypothesis that sensor metadata can provide the basis for robust analytics of “Normal” versus “not Normal” tissue differentiation across various clinical scenarios.

The software development process can involve testing the Temporal Scale Collapsibility (TSC) of the DyPERF time-series curves. This can include the creation of MATLAB-based software capable of generating idealized sinusoidal waveforms with assigned perfusion colors. A development testing platform based on 1 mm² spatial scalability (6,400 RAs) can display color-assigned sinusoidal curves for each RA, with the potential to utilize already-analyzed sensor metadata. Training and testing datasets can be developed from this data to verify the approach, comparing results to both sensor metadata outputs and imaging datasets.

The inventive concepts can extend to the development of a clinical platform compatible with existing software architectures. The TSC algorithms can be tested against specific tissue types and procedures, including gastrointestinal resections, plastic tissue flaps, and vascular limb salvage. This clinical platform can incorporate training data from pre- and post-intervention scenarios or within-field-of-view tissue comparisons to refine predictive differentiation between “Normal” and “not Normal” tissues. Additionally, open-source simulation software capabilities can link TSC outputs to actual LSCI-analyzed imaging data for validation purposes.

Additional aspects of the inventive concepts can address key machine learning considerations. These can include defining “Normal” criteria across multiple episodes, determining the discriminatory delta between “Normal” and “not Normal” tissues, and addressing regions in the field of view where perfusion is not visually apparent due to deeper tissue perfusion. ML processes can be tailored to identify tissue and procedural vulnerabilities to abnormal perfusion patterns while aligning DyPERF analytics with visual discrimination.

Predictive analytics can integrate ML-derived insights into real-time surgical decision-making. These analytics can address questions such as the likelihood of tissue viability, survival probabilities for “not Normal” tissues without further surgical intervention, and comparative analysis of the current intervention against previous cases or aggregated surgical data. Additionally, predictive models can assess wound healing probabilities at various stages of evaluation. Neural network structures, including convolutional neural networks and generative adversarial networks, can be employed to optimize pattern recognition and refine predictive models.

Some inventive concepts can establish a framework for enabling sensor metadata-driven analytics, leveraging advanced machine learning algorithms to provide surgeons with actionable insights and enhance intraoperative decision-making processes. The disclosed software development and predictive analytics systems can represent significant advancements in clinical precision and adaptability.

In some cases, a neural network machine learning environment based on a convoluted neural network structure has been built and is in early testing. A second effort using a generative adversarial network structure to attempt to optimize the pattern recognition is also in development.

In some inventive concepts, converting the 3D block of laser speckle contrast data shown in FIG. 4 (3) into the regional area (RA) analysis image as shown in FIG. 4 (7) opens up novel opportunities to a qualitative analysis of tissue perfusion and viability. For example, the angular directional histogram (FIG. 15) can be formed given a RA image in order to quantify the isotropic pattern of the image which is closely related to the qualitative characteristics of the tissue perfusion. The RA analysis image can be processed even further with the multi-dimensional Fourier transform method which captures the directional and cyclical patterns of the RA image. Then, a computer-vision machine learning platform can be trained to determine the quality level of the tissue perfusion by classifying the Fourier transformed version (2D frequency-domain) of the given RA image.

### Terminology

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may include, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can.” “could.” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for assessing tissue perfusion in a region of interest, the method comprising:
obtaining raw speckle data corresponding to a region of interest, wherein the raw speckle data comprises intensity measurements captured at multiple spatial locations within the region of interest over sequential time points, thereby forming a spatiotemporal series of intensity data for each spatial location;
applying a moving observational window to the temporal series of intensity data for each spatial location to determine intensity-based characteristics for each spatial location over time;
determining a series of speckle contrast values for each spatial location within the region of interest, wherein each speckle contrast value is calculated as a ratio of a mean intensity to a standard deviation of intensity values within the moving observational window, the speckle contrast values collectively forming a spatiotemporal dataset representative of tissue perfusion within the region of interest.

2. The method of claim 1, wherein the intensity-based characteristics comprise intensity values and one or more derivative metrics derived from the intensity values.

3. The method of claim 1, further comprising generating, based on the series of speckle contrast values, a three-dimensional (3D) speckle contrast dataset representing a distribution of speckle contrast values across the spatial locations and sequential time points within the region of interest, wherein the 3D speckle contrast dataset is characterized by a first dimension corresponding to spatial locations along a horizontal axis within the region of interest, a second dimension corresponding to spatial locations along a vertical axis within the region of interest, and a third dimension corresponding to temporal locations determined by sequential time points or frames associated with the speckle contrast values.

4. The method of claim 1, further comprising determining a series of perfusion magnitude values for the region of interest based on the spatiotemporal series of speckle contrast values, wherein the speckle contrast values for all spatial locations within the region of interest and over the temporal sequence of each frame are averaged to produce a single-point perfusion magnitude value for each frame, wherein each perfusion magnitude value corresponds to at least one of the same temporal windows used for determining the series of speckle contrast values, and wherein each perfusion magnitude value is calculated as an average of the speckle contrast values determined for each of the plurality of spatial locations within the region of interest in that corresponding frame.

5. The method of claim 1, further comprising outputting the series of perfusion magnitude values as a time-series curve, wherein the time-series curve is indicative of spatial and/or temporal variations in tissue perfusion within the region of interest.

6. The method of claim 1, further comprising outputting the series of perfusion magnitude values as a scalar mean value calculated over the entirety of the imaging window or one or more subsets thereof, wherein the scalar mean value provides a numeric quantification of perfusion magnitude within the region of interest.

7. The method of claim 4, further comprising analyzing a time-series curve representing the series of perfusion magnitude values to determine one or more physiological parameters associated with tissue perfusion, wherein the physiological parameters include at least one of heart rate, periodicity, or pulsatility, and wherein the analysis further identifies abnormal physiological parameters, including translational motion of the tissues of interest or the imaging system, wherein such motion adversely affects the perfusion magnitude results.

8. The method of claim 7, wherein analyzing the time-series curve to determine the heart rate comprises:
performing a transformation on the time-series curve to decompose it into frequency components;
identifying a frequency component corresponding to oscillations caused by the cardiac cycle; and
calculating the heart rate by converting the identified frequency component into beats per minute.

9. The method of claim 7, wherein analyzing the time-series curve to determine periodicity and/or pulsatility comprises at least one of:
identifying repetitive patterns in the time-series curve based on the intervals between peaks, and calculating periodicity as a measure of the consistency of these intervals over time; or
calculating a magnitude of oscillations in the time-series curve by determining a ratio of peak-to-trough variations relative to a baseline level, and quantifying pulsatility as a measure of the amplitude of the oscillations.

10. The method of claim 5, wherein analyzing the time-series curve to determine pulsatility comprises:
Identifying abnormalities in peak amplitude(s) in the time-series graph that cannot be due to intrinsic perfusion relative to the adjacent perfusion data, and eliminating these abnormalities from the quantitative analyses.

11. The method of claim 2, further comprising comparing perfusion magnitude values derived from distinct temporal points within the temporal domain of the three-dimensional (3D) speckle contrast dataset to identify changes in tissue perfusion, wherein the comparison includes at least one of:
a comparison between perfusion magnitude values before and after an intervention;
an analysis of trends in perfusion magnitude values over successive temporal periods; or
a comparison of perfusion magnitude values against a predefined threshold.

12. The method of claim 11, further comprising generating an output based on the comparison, wherein the output includes at least one of:
a calculated difference or percentage change in perfusion magnitude values between the compared temporal portions;
a time-series graph showing variations in perfusion magnitude values across the temporal portions; or
a binary indicator or temporal markers identifying intervals where a predefined threshold condition is met.

13. The method of claim 2, further comprising segmenting the 3D speckle contrast dataset into a plurality of regional areas, wherein each regional area corresponds to a defined subset of spatial locations within the region of interest, and determining speckle contrast time and space characteristics for each regional area to enable spatially resolved analysis of tissue perfusion.

14. The method of claim 11, further comprising generating an output based on the analysis of the regional areas, wherein the output includes at least one of:
a numerical representation of speckle contrast characteristics for each regional area;
a visual map, graphic, movie or image representing tissue perfusion within the region of interest, segmented by regional areas; or
a comparison of speckle contrast characteristics across the plurality of regional areas to identify spatial variations in tissue perfusion.

15. The method of claim 2, further comprising evaluating the three-dimensional (3D) speckle contrast dataset to identify regions within the region of interest exhibiting variations in perfusion and/or tissue abnormalities, wherein the speckle contrast values correspond to variations in tissue integrity and perfusion at each spatial location, and wherein the identified metrics range from normal tissue integrity with normal perfusion parameters to severely compromised tissue integrity with severely abnormal perfusion parameters.

16. The method of claim 2, further comprising performing a multi-dimensional pattern analysis on the regional areas of the speckle contrast dataset, wherein the multi-dimensional pattern analysis is configured to assess periodicity, directional, and/or anisotropic features of tissue perfusion, and wherein the directional and anisotropic features are identified by applying multi-dimensional Fourier transforms to the patterns of speckle contrast values within the regional areas, identifying the orientation and structural characteristics of the tissue perfusion patterns.

17. The method of claim 14, further comprising generating a directionality histogram based on the multi-dimensional pattern analysis performed on the speckle contrast dataset, wherein the directionality histogram represents the distribution of tissue perfusion within the tissue structures of the region of interest, and wherein the directionality histogram quantifies anisotropic and isotropic components of the tissue perfusion derived from the regional areas of the speckle contrast dataset.

18. A non-transitory computer-readable medium storing instructions that, when executed by a processor, cause a system to perform a method for assessing tissue perfusion in a region of interest, the method comprising:
obtaining raw speckle data corresponding to a region of interest, wherein the raw speckle data comprises intensity measurements captured at multiple spatial locations within the region of interest over sequential time points, thereby forming a spatiotemporal series of intensity data for each spatial location;
applying a moving observational window to the temporal series of intensity data for each spatial location to determine intensity-based characteristics for each spatial location over time; and
determining a series of speckle contrast values for each spatial location within the region of interest, wherein each speckle contrast value is calculated as a ratio of a mean intensity to a standard deviation of intensity values within the moving observational window, the speckle contrast values collectively forming a spatiotemporal dataset representative of tissue perfusion within the region of interest.

19. A system for assessing tissue perfusion in a region of interest, the system comprising:
one or more processors configured to:
obtain raw speckle data corresponding to a region of interest, wherein the raw speckle data comprises intensity measurements captured at multiple spatial locations within the region of interest over sequential time points, thereby forming a spatiotemporal series of intensity data for each spatial location;
apply a moving observational window to the temporal series of intensity data for each spatial location to determine intensity-based characteristics for each spatial location over time; and
determine a series of speckle contrast values for each spatial location within the region of interest, wherein each speckle contrast value is calculated as a ratio of a mean intensity to a standard deviation of intensity values within the moving observational window, the speckle contrast values collectively forming a spatiotemporal dataset representative of tissue perfusion within the region of interest.

20. The system of claim 19, further comprising a memory for storing the spatiotemporal dataset and/or an interface for outputting the spatiotemporal dataset for further analysis or visualization of tissue perfusion.
